(19) United States
(12) Patent Application Publication
(10) Pub. No.: US 2019/0276871 A1
(43) Pub. Date: Sep. 12, 2019

(54) Antimicrobial Cartridges and Processes for Antimicrobial Susceptibility Testing

(71) Applicant: SeLux Diagnostics, Inc., Cambridge, MA (US)

(72) Inventors: Eric Stern, Jamaica Plain, MA (US); Kelly Flentie, Jamaica Plain, MA (US); Aleksandar Vacic, Cambridge, MA (US); Frederick P. Floyd, JR., Boston, MA (US); Sarah A. Scott, Medford, MA (US)

(21) Appl. No.: 15/887,794

(22) Filed: Feb. 2, 2018

Related U.S. Application Data
(60) Provisional application No. 62/454,490, filed on Feb. 3, 2017.
(60) Provisional application No. 62/508,046, filed on May 18, 2017.
(60) Provisional application No. 62/616,800, filed on Jan. 12, 2018.

(51) Int. Cl. C12Q 1/20; C12M 3/00; C12M 1/32; C12M 1/34; C12M 1/36

(52) U.S. Cl. CPC C12Q 1/20; C12M 23/42; C12M 41/48; C12M 41/46; C12M 23/12

(57) ABSTRACT

The present invention provides, among other things, a master cartridge for effective storage and transportation of antimicrobials, the master cartridge comprising multiple reservoir units for placing a plurality of antimicrobials at high concentration from which multiple patient cartridges could be generated for testing a plurality of biological samples, and methods for using the same.

### REFERENCE TO RELATED APPLICATIONS

This application claims the benefit of priority to U.S. Provisional Patent Application No. 62/454,490, filed Feb. 3, 2017, U.S. Provisional Patent Application No. 62/508,046, filed May 18, 2017, and U.S. Provisional Patent Application No. 62/616,800, filed Jan. 12, 2018, the entire disclosures of each of which are hereby incorporated by reference.

### FIELD

The present invention relates generally to antimicrobial susceptibility testing and more specifically to devices and methods for rapid antimicrobial susceptibility testing of clinical samples.

### BACKGROUND

Current broth dilution antimicrobial susceptibility test (AST) methods utilize individual cartridges with less than 130 reservoirs pre-filled with antimicrobial compounds supplied at the desired testing concentrations. Antimicrobial compounds may exhibit poor stability in solution. As a result, cartridges comprising dried antimicrobial compounds are utilized in laboratory practice because they can be shipped and stored at room temperature without antimicrobial degradation. Dried cartridges are designed for reconstitution with aqueous solutions. In order to prevent cross contamination, the AST method relies on transferring the same concentration of a microorganism into each reservoir, such that each cartridge is designed for use with a single microorganism under test. Moreover, each cartridge comes with a preset layout and range of concentration of antimicrobial compounds which limits scope of exploring newer drug concentrations or types for a variety of patient samples. There is therefore a need for more versatile cartridge systems for robust multiplex assay designs. Furthermore, there is a need for increasing numbers of reservoirs per patient cartridge in order to test the larger numbers of antimicrobials available for drug-resistant pathogens.

### SUMMARY

The present invention provides, among other matters, a “master” cartridge useful for preparing a plurality of antimicrobial susceptibility (AST) patient cartridges. Such a cartridge offers a versatile approach to set up robust AST patient cartridge designs. The invention solves an up-and-coming need resulting from the antimicrobial resistance epidemic for testing increased numbers of antimicrobial compounds in parallel for microbial samples derived from patient samples. The master cartridge described herein is suitable for multiple uses and comes with the provision that the multiple uses can be at different times. The invention also provides methods of preparation of a patient test cartridge from the master cartridge described herein, with exemplary methods of use. A master cartridge can be customized towards specific diagnostic and/or therapeutic needs, by suitably allotting the assortments of antimicrobials in an array.

Presently, large numbers of reservoirs on patient cartridges are not available because of engineering limitations of current automated AST platforms that require growth curves to be made with respect to time to deliver accurate results. The invention described herein solves this, and other, problems.

It is understood that any of the aspects and embodiments described below can be combined in any desired way, and that any embodiment or combination of embodiments can be applied to each of the aspects described below, unless the context indicates otherwise.

In one aspect, the invention provides a master cartridge adapted for preparing patient cartridges for antimicrobial susceptibility tests from samples comprising microorganisms, the master cartridge comprising (a) one or more reservoirs; and (b) one or more antimicrobials. The master cartridge comprises sufficient amounts of the one or more antimicrobials for a plurality of independent antimicrobial susceptibility tests performed using a plurality of patient cartridges from a plurality of samples comprising microorganisms.

In some embodiments, the samples in the master cartridge comprising microorganisms are patient-derived.

In some embodiments, the master cartridge is adapted for preparing patient cartridges for antimicrobial susceptibility tests from samples comprising microorganisms, wherein multiple patient cartridges are prepared from a single master cartridge.

In some embodiments, the master cartridge is adapted for preparing patient cartridges for antimicrobial susceptibility tests from samples comprising microorganisms, wherein a patient cartridge contains samples derived from one patient.

In some embodiments, the master cartridge is adapted for preparing patient cartridges for antimicrobial susceptibility tests from samples comprising microorganisms, wherein a patient cartridge contains samples derived from more than one patient.

In some embodiments, the master cartridge comprises antimicrobials that are present in dried or desiccated form or in solvated form.

In some embodiments, the master cartridge comprises antimicrobials wherein each antimicrobial is present in sufficient amount that solvation of the antimicrobial in 0.1 mL of a suitable solvent provides an antimicrobial concentration that is more than 5-fold, or more than 10-fold, or more than 25-fold higher than the highest desired antimicrobial concentration used for antimicrobial susceptibility testing.

In some embodiments, the master cartridge comprises antimicrobials wherein each antimicrobial is present in sufficient amount that solvation of the antimicrobial in 0.1 mL of a suitable solvent provides an antimicrobial concentration that is less than 1000-fold higher than the highest desired antimicrobial concentration used for antimicrobial susceptibility testing.

In some embodiments, the master cartridge the master cartridge comprises antimicrobials wherein each antimicrobial is present in sufficient concentration that is more than 5-fold, more than 10-fold, or more than 25-fold higher than the highest desired antimicrobial concentration used for antimicrobial susceptibility testing.

In some embodiments, the master cartridge comprises antimicrobials wherein each antimicrobial is present in sufficient concentration that is less than 1,000-fold higher than the highest desired antimicrobial concentration used for antimicrobial susceptibility testing.

In some embodiments, the master cartridge comprises antimicrobials wherein a plurality of the antimicrobials are not solubilized

In some embodiments, the one or more non-aqueous solvents are required for antimicrobial solvation.

In some embodiments, the antimicrobials remain stable through multiple freeze-thaw cycles.

In some embodiments, the antimicrobials are frozen in a solvated state below −20° C.

In some embodiments, the master cartridge comprises antimicrobials, wherein the antimicrobials are frozen in a solvated state at about −75° C. to about −80° C.

In some embodiments, the antimicrobial mass per reservoir is ≥0.1, ≥0.5, ≥1, ≥2, ≥5, or ≥10 milligrams.

In some embodiments, the antimicrobial mass per reservoir is ≤1 gram.

In some embodiments, the master cartridge is not designed for contact with microorganisms.

In some embodiments, the master cartridge comprises a larger number of antimicrobials than required for antimicrobial susceptibility testing of any single patient-derived sample comprising microorganisms.

In some embodiments, the master cartridge comprises three or more antimicrobials utilized exclusively for treating infections caused by gram-positive microorganisms and three or more antimicrobials utilized exclusively for treating infections caused by gram-negative microorganisms.

In some embodiments, the master cartridge comprises >1, >2, >5, >10, >15, >20, >25, >30, or >35 antimicrobials.

In some embodiments, the master cartridge comprises <200 antimicrobials.

In some embodiments, the master cartridge is adapted for preparing patient cartridges for antimicrobial susceptibility tests, wherein the patient cartridge comprises 48, 96, 192, 384 or 1536 reservoirs.

In some embodiments, the master cartridge comprises antimicrobials, wherein the one or more antimicrobials are present at sufficient masses to support ≥2, ≥5, ≥10, ≥25, ≥50, or ≥100 independent antimicrobial susceptibility tests of independent samples comprising microorganisms.

In some embodiments, the number of reservoirs in the master cartridge is different from the number of reservoirs on each patient cartridge.

In some embodiments, the number of reservoirs in the master cartridge is less than the number of reservoirs on each patient cartridge.

In some embodiments, the number of reservoirs having an antimicrobial in the patient cartridge is greater than the number of reservoirs having the same antimicrobial in the master cartridge for a plurality of antimicrobials.

In some embodiments, the number of antimicrobial concentrations tested on each patient sample is an integer multiple greater than the number of antimicrobial concentrations present in each master cartridge for a plurality of antimicrobials.

In some embodiments, the number of antimicrobial concentrations tested on each patient sample is at least 2-fold greater than the number of antimicrobial concentrations present in the master cartridge for the plurality of antimicrobials.

In some embodiments, a patient cartridge is prepared from antimicrobials collected from two or more master cartridge.

In some embodiments, the master cartridge comprises antimicrobials, wherein one or more of the antimicrobials are present in a form that it is soluble in dimethyl sulfoxide (DMSO).

In some embodiments, the master cartridge comprises antimicrobials, wherein one or more antimicrobials are solvated in an aqueous solvent.

In some embodiments, the one or more antimicrobials in the master cartridge are solvated in a non-aqueous solvent.

In some embodiments, the master cartridge comprises antimicrobials, wherein one or more antimicrobials are solvated at pH >8.

In some embodiments, the master cartridge comprises antimicrobials, wherein one or more antimicrobials are solvated at pH <7.

In some embodiments, the master cartridge comprises 1, 2, 5, 10 or more reservoirs that do not comprise an antimicrobial.

In some embodiments, the master cartridge comprises antimicrobials, in which one or more of the antimicrobials is selected from the list of FDA-approved antibiotics and/or antifungals.

In some embodiments, the master cartridge comprises antimicrobials, in which one or more of the antimicrobials is selected from a list consisting of: Amikacin, Amikacin-fosfomycin, Amoxicillin, Amoxicillin-clavulanate, Ampicillin, Ampicillin-sulbactam, Azithromycin, Azlocillin, Aztreonam, Aztreonam-avibactam, Besifloxacin, Biapenem, Cadazolid, Carbenicillin, Cefaclor, Cefamandole, Cefazolin, Cefdinir, Cefditoren, Cefepime, Cefepime-tazobactam, Cefetamet, Cefixime, Cefmetazole, Cefonicid, Cefoperazone, Cefotaxime, Cefotetan, Cefoxitin, Ceftolozane-tazobactam, Cefpodoxime, Cefprozil, Ceftaroline, Ceftaroline-avibactam, Ceftazidime, Ceftazidime-avibactam, Ceftibuten, Ceftizoxime, Ceftobiprole, Ceftolozane-tazobactam, Ceftriaxone, Cefuroxime, Cephalothin, Chloramphenicol, Cinoxacin, Ciprofloxacin, Clarithromycin, Clinafloxacin, Clindamycin, Colistin, Dalbavancin, Daptomycin, Delafloxacin, Dirithromycin, Doripenem, Doxycycline, Enoxacin, Eravacycline, Ertapenem, Erythromycin, Faropenem, Fidaxomicin, Finafloxacin, Fleroxacin, Fosfomycin, Fusidic acid, Garenoxacin, Gatifloxacin, Gemifloxacin, Gentamicin, Gepotidacin, Grepafloxacin, Iclaprim, Imipenem, Imipenem-relebactam, Kanamycin, Lefamulin, Levofloxacin, Levonadifloxacin, Linezolid, Linopristin-flopristin, Lomefloxacin, Loracarbef, Mecillinam, Meropenem, Methicillin, Mezlocillin, Minocycline, Moxalactam, Moxifloxacin, Nafcillin, Nalidixic acid, Netilmicin, Nitrofurantoin, Norfloxacin, Ofloxacin, Omadacycline, Oritavancin, Oxacillin, Penicillin, Piperacillin, Piperacillin-tazobactam, Plazomicin, Polymyxin B, Quinupristin-dalfopristin, Razupenem, Rifampin, Solithromycin, Sparfloxacin, Sulfisoxazole, Sulopenem, Tedizolid, Teicoplanin, Televancin, Telithromycin, Tetracycline, Ticarcillin, Ticarcillin-clavulanate, Tigecycline, Tobramycin, Trimethoprim, Trimethoprim-sulfamethoxazole, Trospectomycin, Vancomycin, Aculeacin A, Amphotericin B, Caspofungin, Clotrimazole, Fluconazole, Flucytosine, 5-Fluorocytosine, Griseofulvin, Itraconazole, Ketoconazole, Nystatin, Sordarin, Terbinafine, Vaborbactam-meropenem, Voriconazole and a salt or hydrate form thereof.

In some embodiments, one or more of the antimicrobials in the master cartridge is selected from azoles, echinocandins and polyenes.

In some embodiments, the master cartridge comprises one or more reservoirs that contain more than one antimicrobial.

In some embodiments, the master cartridge comprises one or more reservoirs which comprise one or more optically and/or electrically active chemicals.

In some embodiments, one or more reservoirs of the master cartridge comprise one or more pH indicators.

In some embodiments, one or more reservoirs of the master cartridge comprise one or more optical redox indicators.

In some embodiments, the master cartridge comprises a seal for the one or more reservoirs.

In some embodiments, each reservoir of the master cartridge is independently sealed.

In some embodiments, the master cartridge comprises an outer seal enclosing the cartridge in its entirety.

In some embodiments, the one or more reservoirs in the master cartridge comprise one or more vials and/or matrix vials.

In some embodiments, the master cartridge comprises one or more vials, in which the contents of each vial are sterile.

In some embodiments, the one or more reservoirs of the master comprise a microtiter plate.

In some embodiments, the one or more reservoirs of the master cartridge are sterile.

In some embodiments, the microorganisms tested for antimicrobial susceptibility are bacteria, fungi, protozoa, and/or archaea.

In some embodiments, the microorganisms tested for antimicrobial susceptibility are present in a biological sample.

In some embodiments, the biological sample is processed one or more times, and wherein the processing comprises culturing.

In some embodiments, the biological sample is selected from blood, cerebrospinal fluid, urine, stool, vaginal, sputum, bronchoalveolar lavage, throat, nasal/wound swabs, and combinations thereof.

In some embodiments, the bacteria tested for antimicrobial susceptibility using the master cartridge are selected from the group consisting of Escherichia coli, Enterococcus spp., Staphylococcus spp., Klebsiella spp., Acinetobacter spp., Pseudomonas spp., Enterobacter spp., Streptococcus spp., Proteus spp., Aerococcus spp., Actinomyces spp., Bacillus spp., Bartonella spp., Bordetella spp., Brucella spp., Campylobacter spp., Chlamydia spp., Chlamydophila spp., Clostridium spp., Corynebacterium spp., Ehrlichia spp., Francisella spp., Gardenerella spp., Haemophilius spp., Helicobacter spp., Lactobacillus spp., Legionella spp., Leptospira spp., Listeria spp., Mycobacterium spp., Mycoplasma spp., Neisseria spp., Nocardia spp., Pasteurella spp., Rickettsia spp., Salmonella spp., Shigella spp., Stenotrophomonas spp., Treponema spp., Ureaplasma spp., Vibrio spp., Yersinia spp., and a combination thereof.

In some embodiments, the fungi tested for antimicrobial susceptibility using the master cartridge are selected from the group consisting of Candida spp., Issatchenkia spp., Blastomyces spp., Coccidioides spp., Aspergillus spp., Cryptococcus spp., Histoplasma spp., Pneumocystis spp., Stachybotrys spp., Sporothrix, Exserohilum, Cladosporium, ringworm, mucormycetes, and a combination thereof.

In some embodiments, the master cartridge is adapted for testing quality control for the one or more antimicrobials, which is performed by one or more analytical chemistry methods.

In some embodiments, the master cartridge is adapted for testing quality control, wherein quality control for the one or more antimicrobials is not performed exclusively through microorganism growth.

In some embodiments, the reservoir walls and/or bases of the master cartridge comprise polystyrene.

In some embodiments, the polystyrene is untreated polystyrene.

In one aspect, the inventoin provides a method of using the master cartridge, wherein the method determines antimicrobial susceptibility of one or more microorganisms based on relative microorganism growth.

In one aspect the invention provides a method for preparing a patient cartridge from a master cartridge for antimicrobial susceptibility testing, the method comprising: (a) transferring a plurality of antimicrobials from a master cartridge to a patient cartridge, wherein each antimicrobial in the master cartridge is present in sufficient amount such that solvation of the antimicrobial in 0.1 mL of suitable solvent provides an antimicrobial concentration at least 10-fold higher than the highest desired testing concentration to the patient cartridge; (b) preparing two or more dilutions of each antimicrobial transferred from the master cartridge; (c) inoculating the patient cartridge with a patient sample to one or more reservoirs; and (d) excluding the reservoirs for no-patient-sample negative control.

In some embodiments, two or more patient cartridges are prepared from one master cartridge.

In some embodiments, a patient cartridge is prepared from two or more master cartridges.

In some embodiments, two or more dilutions of each antimicrobial are serial doubling dilutions, wherein an antimicrobial concentration, C, present in a master cartridge reservoir is diluted by a factor d for one patient cartridge reservoir thereby obtaining an antimicrobial concentration C/d, and the C/d concentration is then further diluted two fold for a second patient cartridge reservoir thereby obtaining a concentration of C/(2d), and the process being repeated multiple times to prepare serial dilutions.

In some embodiments, the method comprising adding one or more reagents for antimicrobial susceptibility testing.

In one aspect, the invention provides a method for determining antimicrobial susceptibility of one or more microorganisms using a master cartridge, comprising: (a) performing a plurality of different assays sharing an incubation period, wherein each assay comprises a microorganism growth assay in the presence of one or more antimicrobials, wherein the plurality of different assays are performed on a patient cartridge comprising one or more reservoirs and one or more antimicrobials, wherein the antimicrobials are transferred to the patient cartridge from a master cartridge that contains each antimicrobial present at sufficient mass such that solvation in 0.1 mL of suitable solvent yields an antimicrobial concentration >10-fold higher than the highest desired testing concentration; (b) optionally adding one or more reagents to the patent cartridge for preparing sample dilution, and/or promoting bacterial growth and/or for promoting the antimicrobial susceptibility assay; (c) incubating the patient cartridge for at least 2 hours; and (d) determining antimicrobial susceptibility of the one or more microorganisms based on relative microorganism growth.

In some embodiments, the method comprises determining antimicrobial susceptibility of the one or more microorganisms, which comprises determining a minimum inhibitory concentration (MIC) or a qualitative susceptibility result (QSR) for the one or more antimicrobials.

In some embodiments, the minimum inhibitory concentration (MIC) or the qualitative susceptibility result (QSR) for the one or more antimicrobials is determined from a plurality of assays.

In some embodiments, the number of assays used to determine the minimum inhibitory concentration (MIC) or the qualitative susceptibility result (QSR) for the one or more antimicrobials is smaller than the number of assays performed.

In some embodiments, the number of assays used to determine the minimum inhibitory concentration (MIC) or the qualitative susceptibility result (QSR) for the antimicrobial is equal to the number of assays performed.

In some embodiments, the method comprising determining whether an assay is appropriate for determining the one or more microorganism's susceptibility to the one or more antimicrobials.

In some embodiments, a different assay is used for different antimicrobial-microorganism combinations.

In some embodiments, the patient cartridge antimicrobials are derived from two or more independent master cartridges.

In some embodiments, the one or more of the antimicrobials and/or dilution ranges transferred from a single master cartridge is different for different patient cartridges.

In some embodiments, the one or more antimicrobial solutions or one or more antimicrobial dilution ranges are different for different microbial species tested.

In some embodiments, the antimicrobials in the patient cartridge comprising patient sample are different from the dilution ranges of antimicrobials in patient cartridges for quality control of the AST process.

In some embodiments, a plurality of the antimicrobial solutions from a master cartridge reservoir are transferred to two or more patient cartridge reservoirs.

In some embodiments, one or more reagents for one or more reservoirs in the patient cartridge are not present on any master cartridge.

In some embodiments, the method is one, wherein an automated liquid handler is used, wherein the automated liquid handler comprises 24-, 48-, 96-, or 384-well manifold liquid handling head compatible with microtiter plates to transfer antimicrobial solutions from master cartridges to patient cartridges.

In some embodiments, the reservoir walls and/or bases of the patient cartridge comprise polystyrene.

In some embodiments, the polystyrene is untreated polystyrene.

In some aspects, the invention provides a patient cartridge for performing antimicrobial susceptibility testing comprising at least 150 reservoirs for testing a plurality of antimicrobials, wherein the dilution range for a plurality of antimicrobials in the cartridge exceeds the clinically relevant dilution range by at least one dilution.

In some embodiments, the patient cartridge comprises less than 3,000 reservoirs.

In some embodiments, the patient cartridge comprises 384 reservoirs.

In some aspects, the invention provides a patient cartridge for automated antimicrobial susceptibility testing of sterile and non-sterile patient samples comprising gram-negative bacteria, the cartridge comprising at least 150 reservoirs and ≥20 different antimicrobials comprising amikacin, ampicillin-sulbactam, amoxicillin-clavulanate, aztreonam, cefazolin, cefepime, ceftolozane-tazobactam, ceftazidime, ceftazidime-avibactam, ceftriaxone, cefepime, cefoxitin, ciprofloxacin, ertapenem, gentamicin, levofloxacin, meropenem, piperacillin-tazobactam, tetracycline, tobramycin, and trimethoprim-sulfamethoxazole, wherein the patient cartridge provides at least the clinically relevant dilution ranges for a plurality of antimicrobials known to be effective against Enterobacteriaceae and Pseudomonas spp. derived from sterile and non-sterile patient samples.

In some embodiments, the patient cartridge comprises at least the clinically relevant dilution ranges for a plurality of antimicrobials known to be effective against Acinetobacter spp. from sterile and non-sterile patient samples.

In some aspects, the invention provides a patient cartridge for inoculation with a gram-positive bacteria sample derived from a human sample for automated antimicrobial susceptibility testing comprising ≥150 reservoirs and ≥20 different antimicrobials comprising, azithromycin, ceftaroline, clindamycin, ciprofloxacin, daptomycin, gentamicin, levofloxacin, linezolid, minocycline, oxacillin, tetracycline, trimethoprim-sulfamethoxazole, and vancomycin, such that the patient cartridge provides clinically relevant dilution ranges for a plurality of antimicrobials known to be effective against Staphylococcus spp. and Enterococcus spp. derived from patient samples from sterile and non-sterile patient samples.

In some aspects the invention provides a patient cartridge for inoculation with a bacteria sample derived from a human sample for automated antimicrobial susceptibility testing comprising ≥150 independent reservoirs and ≥25 different antimicrobials comprising amikacin, ampicillin-sulbactam, aztreonam, cefepime, ceftazidime, ceftazidime-avibactam, ceftriaxone, ciprofloxacin, daptomycin, gentamicin, levofloxacin, linezolid, meropenem, piperacillin-tazobactam, tetracycline, tobramycin, trimethoprim-sulfamethoxazole, and vancomycin, such that the patient cartridge provides clinically relevant dilution ranges for a plurality of antimicrobials known to be effective against Enterobacteriaceae, Pseudomonas spp., Acinetobacter spp., Staphylococcus spp., and Enterococcus spp. derived from patient samples from sterile and non-sterile patient samples.

In some embodiments, the patient sample is urine, blood, cerebrospinal fluid, synovial fluid, aspirate, respiratory, or wound swab.

In some embodiments, the patient cartridge comprises antimicrobials, wherein the suitable antimicrobial dilution ranges include the range suitable for testing bacteria from urine samples.

In some embodiments, the patient cartridge further comprises at least three reservoirs with no reagents; and/or at least three reservoirs having no reagent that affects microbial growth.

In some aspects, the invention provides a patient cartridge for automated antimicrobial susceptibility testing suitable for inoculation with a microbial sample derived from a human sample, the cartridge comprising ≥150 reservoirs and ≥20 different antimicrobials, wherein: (a) at least one assay quality control, where three or more reservoirs comprise no reagents; (b) at least one assay quality control having three or more reservoirs comprising no reagents that influence microorganism growth; and (c) the dilution ranges of at least 5 antimicrobials exceed the clinically relevant dilution ranges for the bacterial species by at least one antimicrobial concentration.

In some embodiments, the patient cartridge comprises amikacin, ampicillin-sulbactam, aztreonam, cefepime, cefotaxime, ceftazidime, ceftazidime-avibactam, ceftriaxone, ciprofloxacin, gentamicin, imipenem, levofloxacin, meropenem, piperacillin-tazobactam, tetracycline, tobramycin, and trimethoprim-sulfamethoxazole.

In some embodiments, the dilution ranges of a plurality of antimicrobials are clinically relevant for Enterobacteriaceae, Pseudomonas spp., and Acinetobacter spp.

In some embodiments, the patient comprises ciprofloxacin, daptomycin, gentamicin, levofloxacin, linezolid, penicillin, tetracycline, and vancomycin.

In some embodiments, the dilution ranges of a plurality of antimicrobials are clinically relevant for Staphylococcus spp. and Enterococcus spp.

In some embodiments, the patient cartridge comprises a microtiter plate comprising 384 reservoirs.

In some embodiments, the patient cartridge comprises a microtiter plate comprising 1536 reservoirs.

In some embodiments, each reservoir comprises reservoir wall and a reservoir base, and wherein the reservoir walls for a plurality of reservoirs are opaque.

In some embodiments, a plurality of the reservoirs allow >85% passage of light at 350 nm through the reservoir bases.

In some embodiments, the reservoir walls and/or bases of patient cartridge comprise polystyrene.

In some embodiments, the polystyrene is untreated polystyrene.

In some embodiments, the patient cartridge comprises antimicrobials in solid state.

In one aspect, the invention provides a pouch comprising a patient cartridge of and a desiccant, wherein the patient cartridge is sealed within the pouch comprising the desiccant.

In some embodiments, the patient cartridge is stable for storage between 0° C. and 35° C.

In some embodiments, the patient cartridge comprises antimicrobials which are frozen in solvated form.

In some embodiments, the pouch comprises a patient cartridge and an adhesive cover, wherein the patient cartridge is sealed with the adhesive cover.

In some embodiments, the patient cartridge further comprises a detachable lid.

In some embodiments, the antimicrobial amounts in a plurality of reservoirs are replicated in one or more additional reservoirs.

The invention provides a method for automated antimicrobial susceptibility testing comprising: (a) preparing a patient cartridge comprising about 384 reservoirs, wherein a first subset of the 384 reservoirs comprises one or more antimicrobials, by inoculating a second subset of the 384 reservoirs with a microorganism-comprising sample, and providing within the patient cartridge a plurality reservoirs for assay quality control, comprising a minimum of 3 reservoirs for negative control having no reagents; and a minimum or 3 reservoirs having no reagent that promote bacterial growth; (b) incubating the cartridge under conditions promoting microorganism growth for a period between 2 and 24 hours; (c) interrogating a plurality of reservoirs to assess microbial growth; and (d) determining the MIC for the sample for a plurality of antimicrobials on the cartridge.

In some embodiments, a plurality of reservoirs on the cartridge are interrogated for growth between 1 and 5 times before the MIC is determined.

In some embodiments, the method of claim 113, wherein a plurality of reservoirs on the cartridge are interrogated for growth between 1 and 4 times before the MIC is determined.

In some embodiments, a plurality of reservoirs on the cartridge are interrogated for growth between 1 and 3 times before the MIC is determined.

In some embodiments, no more than 98% of the reservoirs are utilized to provide MIC results.

In some embodiments, a minimum of 3 reservoirs are utilized to determine the incubation period when sufficient microbial growth has been achieved to initiate one or more assays for AST.

In some embodiments, one or more reagents are added to the patient cartridge after the incubation period.

In some embodiments, the concentration of patient sample inoculated in a reservoir within the second subset of reservoirs is different from the concentration of patient samples inoculated in a different reservoir within the same subset in the patient cartridge.

In some embodiments, the lid, the pouch and/or the adhesive cover is removed prior to inoculating.

In some embodiments, the method describes comprises adding a chemical reagent solutions in a plurality of the reservoirs, wherein the chemical reagents solution comprise a molecule capable of undergoing a chemical reaction.

### BRIEF DESCRIPTION OF THE DRAWINGS

The above and further features will be more clearly appreciated from the following detailed description when taken in conjunction with the drawings. The drawings are however for illustration purpose only, not for a limitation.

FIG. 1 depicts space requirements for AST master cartridge versus available AST assay plates. Compared to the 50 assay plates, master cartridge of three 96 well plates require considerably less storage space.

FIG. 2 depicts layout of antimicrobials on a three plate master cartridge comprising an antimicrobial panel known to act against both gram positive and gram negative bacteria; an antimicrobial panel known to against gram negative bacteria; and an antimicrobial panel known to be act against gram positive bacteria. Each antimicrobial is depicted by a three letter abbreviation of the convention.

FIG. 3 depicts a schematic diagram of the inoculation workflow for a master cartridge AST assay.

FIG. 4 depicts an individual well flow chart for setting up a patient cartridge from a master cartridge for an AST assay.

FIG. 5 depicts the final layout of a patient cartridge 384 well comprising antimicrobials against gram negative bacteria.

FIG. 6 depicts the final layout of a patient cartridge 384 well comprising antimicrobials against gram positive bacteria.

FIG. 7 depicts bacterial growth results showing minimum inhibitory concentration (MIC) values for each antimicrobial. Graphs in the top row contain data from 384-well plates. Graphs in the bottom row contain data from 96-well plates.

FIG. 8 depicts percentage of volume losses during an AST assay in 384-well plates from two different experiments (left panel) and from central vs. edge wells on the 384-well plate (right panel).

FIG. 9A-B depicts patient cartridge Broad Spectrum plate layout.

FIG. 10A-B depicts patient Cartridge layout of Gram positive or Gram negative antimicrobials.

### DEFINITIONS

The patent and scientific literature referred to herein establishes knowledge that is available to those of skill in the art. The issued U.S. patents, allowed applications, published foreign applications, and references that are cited herein are hereby incorporated by reference to the same extent as if each was specifically and individually indicated to be incorporated by reference.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

As used herein, unless specifically indicated otherwise, the word “or” is used in the inclusive sense of “and/or” and not the exclusive sense of “either/or.”

In order for the present invention to be more readily understood, certain terms are first defined below. Additional definitions for the following terms and other terms are set forth throughout the specification.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal are transgenic animals, genetically-engineered animals, and/or a clone.

Antimicrobial: As used herein an antimicrobial refers to an agent that kills (microbicidal), attenuates (microbistatic) or inhibits the function of a microorganism. An antimicrobial can be a chemical compound, a biological product, such as a peptide, protein, an antibody or a nucleic acid, or a small molecule. It may be naturally occurring product or a synthetic product.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Clinical breakpoint ranges for various antimicrobials are provided in the Clinical and Laboratory Standards Institute (CLSI) publication “M100—Performance Standards for Antimicrobial Susceptibility testing,” the FDA website at https://www.fda.gov/Drugs/DevelopmentApprovalProcess/DevelopmentResources/ucm575163.h tm, and the EUCAST website http://www.eucast.org/clinical_breakpoints/. This set of values determines the interpretive criteria of the MIC result determined by the AST assay. All MIC values up to and including the susceptible value will be reported as Susceptible to the clinical floor. For MICs above the Susceptible value, depending on the antimicrobial and species under test, values of Intermediate, Susceptible Dose-Dependent, and Resistant may be reported. For example, for ciprofloxacin and Enterobacteriaceae, the Susceptible MIC cutoff is 1 μg/mL; an MIC of 2 μg/mL is reported as Intermediate; and all MICs above 4 μg/mL are reported as Resistant.

Clinically relevant dilution range: As used herein, a “clinically relevant dilution range” is the clinical breakpoint range plus two dilutions below the Susceptible value and one dilution above the Resistant value. For example, for ciprofloxacin and Enterobacteriaceae, the Susceptible MIC cutoff is 1 μg/mL and all MICs above 4 μg/mL are reported as Resistant, so the clinically relevant dilution range would span from 0.25 μg/mL to 8 μg/mL.

Delivery: As used herein, the term “delivery” encompasses both local and systemic delivery. For example, delivery of antimicrobial encompasses situations in which an antimicrobial is delivered to a target tissue and the encoded protein is expressed and retained within the target tissue (also referred to as “local distribution” or “local delivery”), and situations in which an antimicrobial is delivered to a target tissue and the encoded protein is expressed and secreted into patient's circulation system (e.g., serum) and systematically distributed and taken up by other tissues (also referred to as “systemic distribution” or “systemic delivery).

Dilution range: As used herein, dilution range refers to range of serial dilutions (or “doubling” dilutions) for a given antimicrobial, such as is standard for broth microdilution AST. For example, for a representative antimicrobial, such as ciprofloxacin, this range may comprise the dilutions: 16 μg/mL, 8 μg/mL, 4 μg/mL, 2 μg/mL, 1 μg/mL, 0.5 μg/mL, 0.25 μg/mL, 0.125 μg/mL, etc. Serial dilution may refer to dilutions by a factor other than 2 (doubling dilution). In certain instances, serial dilutions may be performed by a dilution factor of 5, or a dilution factor of 10 in order to cover the minimum and maximum range desirable within the number of dilutions. However, for the purpose of examples described herein, unless otherwise indicated, a dilution factor is 2.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Master cartridge, patient cartridge, test cartridge: As used herein, master cartridge is the parent cartridge from which daughter “patient” or “sample” cartridges are prepared by dispensing antimicrobial compounds from the master cartridge to the daughter patient cartridges. In some embodiments daughter cartridges have serial dilutions of antimicrobial compounds, whereas the master cartridge comprises the concentrated or lyophilized form of the antimicrobial compounds. As used herein, patient cartridge, daughter cartridge, test cartridge, sample cartridge, or sample test cartridge are used interchangeably, which are distinct from the master cartridge.

Microorganism: As used herein, a microorganism is an organism such as bacteria, a virus, protozoa, algae, fungi or any microbial agent which can cause a disease in a human or an animal subject. A microorganism may also remain latent for indefinite period of time in a subject and may not ever cause a disease.

Minimum inhibitory concentration (MIC): As used herein, the MIC of an antimicrobial refers to the lowest concentration of the antimicrobial at which concentration its antimicrobial activity is detectable.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Reservoir: As used herein the term reservoir is used to represent a housing space for holding a composition, such as a reagent or a sample; for storage, or for preparation of, or for performing an assay. The term may be used interchangeably with “wells” for example, in a cartridge or a multi-well microtiter plate. A reservoir may be a single well structure. The reservoir may also be in any form and shape, including but not limited to round wells, or wells of any shape or size, or elongated channels. A reservoir is meant to hold a fluid or dried/lyophilized powder substance.

Sample: As used herein, the term “sample” refers to a biological sample, a patient sample, or a microorganism-containing sample.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but the subject may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target microbe: As used herein, a target microbe is a microbe against which the antimicrobial in question is effective as a microbicidal, microbistatic or inhibitory agent to disrupt a certain function of the microbe relating to its infectivity.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Qualitative Susceptibility Result (QSR): As used herein, the QSR refers to a determination whether or not an antimicrobial has an effect on a microbe, and whether a microbe is susceptible to the antimicrobial and vice versa. For example, the microbe stops growth in presence of the antimicrobial, is an indication that the antimicrobial has an effect on the microbe.

### DETAILED DESCRIPTION

The present invention provides, among other things, a master cartridge for preparing patient cartridges useful for conducting multiplex antimicrobial susceptibility testing (AST) assays. A master cartridge comprises one or more reservoirs having one or more antimicrobials. The master cartridge facilitates creating multiple patient cartridges (i.e., daughter cartridges), wherein the patient cartridges are used for performing one or more multiplex assays for antimicrobial susceptibility. The patient cartridge has greater number of reservoirs having antimicrobials than that of a master cartridge. The patient cartridges or daughter cartridges are dispensable after the test has been performed, whereas, the master cartridge is reusable over a plurality of such test sets, i.e., the master cartridge can be used to prepare a plurality of daughter or patient cartridges. In another aspect, the invention provides a patient cartridge having greater than 150 reservoirs comprising one or more antimicrobials. The invention provides a versatile system to test greater number of antimicrobials and/or greater range of concentrations of the antimicrobials, which could be customized for a patient's needs.

### Disadvantages of Existing AST Platforms

A significant shortcoming of current automated phenotypic antibiotic susceptibility testing (AST) platforms is their inability to accommodate newly-approved antibiotics on their menus, resulting in an average of a 5-year delay between new drug approval and presence on automated AST menus. This poses a significant problem for Infectious Disease (ID) patient care because new antibiotics are often more effective and less toxic than generic alternatives and ID doctors cannot confidently prescribe targeted antibiotic therapies without AST results.

Thus, new, highly-effective antibiotics are often underutilized, resulting in increases in mortality and hospital costs, the latter primarily due to increased lengths-of-stay. This also harms Antibiotic Stewardship Program goals, which aim to deliver the most appropriate antibiotic therapy to each patient as quickly as possible. Furthermore, this delay decreases incentives to pharmaceutical companies to develop new antibiotics, a grave international concern given the current antibiotic resistance epidemic.

Phenotypic AST provides the key actionable information to physicians to determine the proper antibiotic therapy by determining the ability of each of a panel of antibiotics to inhibit bacterial growth. This is most commonly determined by broth microdilution (BMD), a method that determines minimum inhibitory concentrations (MICs) for each of a panel of antibiotics for a patient sample. In order to determine an accurate MIC for a given antibiotic, a range of concentrations must be tested. Thus, AST “panels” comprise multiple antibiotics, each tested at a range of concentrations, with each “well” having an antibiotic at a given concentration.

There are three fully-automated phenotypic AST platforms that dominate the clinical laboratory market, the bioMerieux Vitek2®, the Danaher MicroScan®, and the Becton-Dickinson Phoenix®, and one new rapid-AST entrant, the Accelerate Diagnostics Pheno®. Each of these systems performs phenotypic AST determinations by measuring growth of all wells in their panels repeatedly, such as every 15-30 minutes. Results are then reported when the systems' algorithms determine that sufficient delineation between growth and inhibition is available for each antibiotic to make an accurate MIC call.

Although existing AST platforms can provide accurate results, their reliance on repeated measurements places a significant engineering limitation on the number of antibiotics that can be tested in parallel. Thus, these platforms are limited to menus of less than 20 antibiotics (1-14 for the Pheno®, depending on the organism). This limited space provides clinical lab customers with very limited choice of antibiotics to include on their panels: since new antibiotics often cost more than 10 times per dose compared to generics, customers often are forced to forego these in lieu of the more cost-effective options.

In contrast, the Centers for Laboratory Standards Institute (CLSI) BMD reference method, the “gold standard” phenotypic AST method, performs a single, optical read after an incubation of 16-20 hours. This method thus trades off time for simplicity, with only a single, “endpoint” read necessary. In some instances, the method relies on visual (by-eye) interpretation of results. The current provisions allow limited antimicrobial panels occupying 96-well plates.

By emulating the endpoint assay paradigm of the CLSI reference method, the present method enables greater than 150 reservoirs or wells to be multiplexed by removing the engineering pressure to reduce the number of wells per panel. In some embodiments, the present method enables greater than 200 wells for multiplex assays. As described in U.S. Pat. No. 9,834,808, the assay provides accurate AST data after only 3.5-hour incubations. In order to accommodate slow-growing strains, such as vancomycin-intermediate Staphylococcus aureus (VISA), the method measures <5 wells per panel to ensure that a “sufficient growth” threshold has been reached in order to begin assay processing. In particular, this allows standard microplate formats of 384 or 1536 wells to be used, and it further enables parallel processing of panels with any number of wells greater than 200.

### Multiplex Assays

By running large numbers of multiplex assays in parallel per patient sample, the present platform is able to address three specific user requirements: first, that large numbers of antibiotics, including recently-approved drugs, be available on standard panels; second, that “full” dilution series be utilized; and third, that accuracy is increased by performing replicate tests around breakpoint regions.

In one embodiment, each patient sample can be tested with greater than or equal to 3 antimicrobials in parallel. In one embodiment, each patient sample can be tested with greater than or equal to 4, 5, 6, 7, 8, 9, 10 ,11, 12, 13, 14, 15, 16, 17, 18, 19, 20, 21, 22, 23, 24, 25, 26, 27, 28, 29 or greater than or equal to 30 antimicrobials in parallel. For Gram-negative organisms, in particular, each patient sample can be tested with greater than or equal to 35 antibiotics in parallel. In addition to testing all standard generic antibiotics of all classes, these may include newly-approved and yet-to-be-approved antibiotics including, but not limited to: Avycaz, Vabomer, Zerbaxa, Tedizolid, Tigecycline, Doripenem, Delafloxacin, Oritavancin, Telavancin, Dalbavancin, Eravacycline, Cefiderocol, Omadacycline, Plazomicin, Iclaprim, Lefamulin, Solithera, Primaxin, SPR-994, and MK-7655.

The large number of wells that can be run in parallel further enables large dilution ranges to be tested. As known to those skilled in the art, the CLSI standard is to run serial (or two-fold) dilution ranges of each antibiotic to accurately determine the MIC. The ranges include the “breakpoint” range, the MIC value(s) at which the FDA and CLSI determine that the drug will be clinical effective (“susceptible, S”) or ineffective (“resistant, R”). For example, a drug such as oxacillin with Staphylococcus aureus, an MIC of 2 μg/mL or lower is interpreted to mean the strain is susceptible and the drug should be used, whereas an MIC of 4 μg/mL and higher means the organism is resistant and would be clinically ineffective. Since there are no dilutions between these test wells, most drugs, such as Ertapenem with Escherichia coli, have an additional, “intermediate,” breakpoint to provide an intermediate, buffer region, where clinical use is generally dependent upon breakpoints to other drugs. An exemplary breakpoint table for commonly used antimicrobials known to be effective against Enterobacteriaceae and P. aeruginosa are provided in Table 1. Additional information may be accessed from the following references : Clinical and Laboratory Standards Institute (CLSI) publication “M100—Performance Standards for Antimicrobial Susceptibility testing,” the FDA website at https://www.fda.gov/Drugs/DevelopmentApprovalProcess/DevelopmentResources/ucm575163.htm, and the EUCAST website http://www.eucast.org/clinical_breakpoints.

TABLE 1
FDA Breakpoints for determining susceptibility or resistance to
antimicrobials for two exemplary bacteria.
FDA Breakpoints (≤S | I | ≥R)

| Drug | Enterobacteriaceae | | | P. aeruginosa | | |
|---|---|---|---|---|---|---|
| Amikacin | 16 | 32 | 64 | 16 | 32 | 64 |
| Amoxicillin- | 8/4 | 16/8 | 32/16 | | | |
| Clavulanic Acid | | | | | | |
| Ampicillin | 8 | 16 | 32 | | | |
| Ampicillin- | 8/4 | 16/8 | 32/16 | | | |
| Sulbactam | | | | | | |
| Azithromycin | | | | | | |
| Aztreonam | 4 | 8 | 16 | 8 | 16 | 32 |
| Cefazolin | 1 | 2 | 4 | | | |
| Cefepime | 2 | 4-8 | 16 | 8 | | 16 |
| Cefotaxime | 1 | 2 | 4 | | | |
| Cefoxitin | 4 | 8 | 16 | | | |
| Cefpodoxime | 2 | 4 | 8 | | | |
| Ceftaroline | 0.5 | 1 | 2 | | | |
| Ceftazidime | 4 | 8 | 16 | 8 | | 16 |
| Ceftazidime- | 8/4 | | 16/4 | 8/4 | | 16/4 |
| Avibactam | | | | | | |
| Ceftolozane- | 2/4 | 4/4 | 8/4 | 4/4 | 8/4 | 16/4 |
| Tazobactam | | | | | | |
| Ceftriaxone | 1 | 2 | 4 | | | |
| Cefuroxime | 8 | | 16 | | | |
| Ciprofloxacin | 1 | 2 | 4 | 1 | 2 | 4 |
| Clindamycin | | | | | | |
| Colistin | | | | ~2 | ~4 | ~8 |
| Dalbavancin | | | | | | |
| Daptomycin | | | | | | |
| Delafloxacin | 0.25 | 0.5 | 1 | 0.5 | 1 | 2 |
| Doripenem | | | | 2 | | |
| Doxycycline | | | | | | |
| Ertapenem | | | | | | |
| Erythromycin | | | | | | |
| Gentamicin | 4 | 8 | 16 | 4 | 8 | 16 |
| Imipenem | 1 | 2 | 4 | 2 | 4 | 8 |
| Levofloxacin | 2 | 4 | 8 | 2 | 4 | 8 |
| Linezolid | | | | | | |
| Meropenem | 1 | 2 | 4 | 2 | 4 | 8 |
| Minocycline | 4 | 8 | 16 | | | |
| Moxifloxacin | 2 | 4 | 8 | | | |
| Nitrofurantoin | 32 | 64 | 128 | | | |
| Oritavancin | | | | | | |
| Oxacillin | | | | | | |
| Penicillin | | | | | | |
| Piperacillin- | 16 | 32-64 | 128 | 16 | 32-64 | 128 |
| Tazobactam | | | | | | |
| Quinupristin- | | | | | | |
| Dalfopristin | | | | | | |
| Rifampin | | | | | | |
| Tedizolid | | | | | | |
| Tetracycline | 4 | 8 | 16 | | | |
| Tigecycline | 2 | 4 | 8 | | | |
| Tobramycin | 4 | 8 | 16 | | | |
| Trimethoprim- | 2/38 | | 4/76 | | | |
| Sulfamethoxazole | | | | | | |
| Vabobactam- | 4/8 | 8/8 | 16/8 | | | |
| Meropenem | | | | | | |
| Vancomycin | | | | | | |

With the evolution of microbes and emergence of newer antibiotic resistant varieties of microbes, the standard preset of antimicrobials for AST fall short to meet the requirements for addressing and identifying the antimicrobial that would best fit each patient to treat an infection. The invention is based, in part on a surprising discovery that the ranges of antimicrobials beyond clinical dilution ranges can prove to be advantageous. This necessitates increasing the antimicrobial dilution ranges tested to include dilutions beyond the clinical dilution range. In another embodiment, the invention addresses the need for evaluating slow growing microbes in response to certain antimicrobials or certain concentrations of antimicrobials, such that a perceived positive result of an AST is properly validated, and can provide additional insight into ultimate clinical efficacy of the antimicrobial. The versatility offered by the multiplexing platform disclosed here, offers the advantage of testing not only a greater number of antimicrobials but also a greater range of antimicrobial dilutions. The multiplexing platform offers the ability to customize a particular set of tests as per the requirement of the patient, the disease symptoms and any other relevant factors.

Guidelines for selection of antimicrobials for testing and reporting antimicrobial susceptibility can be had from FDA resources, such as the CLSI M100 guide, the FDA website https://www.fda.gov/Drugs/DevelopmentApprovalProcess/DevelopmentResources/ucm575163.h tm. Similarly, recommendations for testing conditions, routine quality control recommendations, suggestions for additional agents that should be considered for routine testing and reporting can also be obtained from the CSLI guide cited above.

An expanded number of wells is able to test each dilution concentration in duplicate, in triplicate or in greater number of replicates, or to test intermediate dilution concentrations (such as 3 μg/mL), and/or to extend the dilution ranges per antibiotic. These may provide greater accuracy and/or information into susceptibility and/or resistance.

An additional advantage of patient cartridges with >200 reservoirs or wells is that multiple patient samples can be processed on a single plate for cases known by those skilled in the art to be “simple,” such as uncomplicated urinary tract infections. These cases may require parallel testing with smaller number of antibiotics; thus, to conserve cost and time, it may be beneficial to run multiple samples per single cartridge.

In some embodiments, the patient cartridges with >200 reservoirs is used to accommodate multiple samples collected from the same patient, for example body fluid samples such as blood, CSF, serum, pulmonary lavage, saliva or urine. In general, some samples are collected under aseptic conditions such samples are referred to as sterile samples. For some samples, it is not possible to maintain aseptic conditions, such samples are referred to as nonsterile. A patient cartridge of greater than 200 reservoirs allows testing both sterile and nonsterile samples in the same cartridge, given the possibility of avoiding cross contamination from the two kinds of samples being in adjacent reservoirs.

### Master Cartridges

The current standard in automated phenotypic AST is for each patient sample to be tested on a cartridge that is delivered to the laboratory (from the test supplier) with all necessary antibiotics for all dilutions present in the required amounts. This puts the responsibility for accurate antibiotic measurements solely in the factory floor of the manufacturers. The antibiotics are often dried, stabilizing them, such that they may be stored at room temperature or under refrigeration, a significant advantage over frozen (−20° C.) or deep-frozen (−80° C.) storage primarily because of cost. However, it is well known to those skilled in the art that the drying process can have detrimental impacts on antibiotic performance, which may compromise product and result in recalls.

As disclosed herein, the master cartridge is often a single “master” plate which comprises multiple reservoirs, the reservoir comprising antimicrobials in sufficient quantities so as to provide for setting up antimicrobial susceptibility tests (AST) for multiple patient samples and over a range of antimicrobial concentrations. In some embodiments, a single master cartridge enables testing of greater than 25 independent patient samples. The same master cartridge can accommodate a plurality of antimicrobials at quantities or concentrations sufficient for preparing a plurality of antimicrobial susceptibility tests for a plurality of patient samples and multiple reiterations of the same for obtaining confidence in the results. This vastly reduces required storage space, and this may enable antibiotics to be provided to laboratory customers in a frozen or deep-frozen format, which may result in improved batch to catch consistency. Alternatively, the antibiotics may be dried or lyophilized and stored at room temperature or under refrigeration. In some embodiments a master cartridge comprises both individual antimicrobials and antimicrobial combinations. One or more reservoirs in the master cartridge can harbor a combination of more than one antimicrobial compounds.

Accordingly a master cartridge comprises a plurality of reservoirs. In some embodiments the master cartridge comprises 384 or more reservoirs. This allows for introduction of a sufficient number of antimicrobials, including recently approved ones, which is not feasible with 96-reservoir cartridges. It further allows for customization of the antimicrobial panel on each patient plate.

In some embodiments, the master cartridge is designed such that it can undergo multiple freeze thaw cycles without any damage or loss of activity of the antimicrobial compounds. In some embodiments the master cartridge is capable of withstanding extreme temperatures such below −80° C. and can be maintained without undergoing structural damage, such as cracking or warping over a wide range of temperatures.

In some embodiments, the master cartridge comprises one or more encasements or seals. An outer seal or encasement may be present which serves to isolate the cartridge from contamination prior to use. This is useful for transportation and storage of the cartridge. In some embodiments the reservoirs are sealed by another encasement. In some embodiments each reservoir is individually sealed. In some embodiments, each reservoir is sealed by an airtight covering. Additionally each reservoir seal may be individually operable. In some embodiments the encasement is a pouch, which is sealed. In some embodiments the sealed pouch comprises a master cartridge. A master cartridge comprising antimicrobials in solid form is sealed in presence of a desiccant inside the pouch, to keep it dehydrated. Therefore, a pouch comprising a master cartridge and a desiccant is used to seal a master cartridge comprising antimicrobials in solid state. Further, a master cartridge comprising antimicrobials in a solvated form can sealed with an adhesive sealer and/or stored or shipped inside the pouch.

In some embodiments the master cartridge is transparent. In some embodiments the master cartridge is light protected. In some embodiments the master cartridge allows light to penetrate through the base of the reservoirs.

In some embodiments, the master cartridge comprises matrix tubes.

In some embodiments, a master cartridge provides sufficient antimicrobials to prepare 50-100, 100-250, 250-500, 600-750 or 750-1,000 patient cartridges or microtiter plates.

In some embodiments, the master cartridge comprises at least 10 fold higher amount of each antimicrobial required for the highest desired testing concentration in a patient cartridge. In some embodiments, the master cartridge comprises at least 20 fold higher amount of each antimicrobial required for the highest desired testing concentration in a patient cartridge. In some embodiments, the master cartridge comprises at least 30, at least 40, at least 50, at least 60, at least 70, at least 80, at least 90, at least 100 fold higher amount of each antimicrobial required for the highest desired testing concentration in a patient cartridge. In some embodiments, the master cartridge comprises at least 200 fold higher amount of each antimicrobial required for the highest desired testing concentration in a patient cartridge. In some embodiments, the master cartridge comprises at least 500 fold higher amount of each antimicrobial required for the highest desired testing concentration in a patient cartridge. In some embodiments, the master cartridge comprises at least 1,000 fold higher amount of each antimicrobial required for the highest desired testing concentration in a patient cartridge. In some embodiments, the master cartridge comprises at least 10,000 fold higher amount of each antimicrobial required for the highest desired testing concentration in a patient cartridge. In some embodiments the master cartridge comprises as high as 10⁶ fold higher the amount of each antimicrobial required for the highest desired testing concentration in a patient cartridge. In some embodiments, the antimicrobials in the master cartridge are in lyophilized or otherwise dried solid form. In such embodiments, it is necessary to solubilize or solvate the solid form into a high concentration stock solution for each antimicrobial to aliquot a fraction of the solution into a patient cartridge or an auxiliary reservoir or dilution reservoir. A number of serial dilutions can be generated from the master cartridge for a patient cartridge.

In some embodiments the antimicrobials are in solution in a master cartridge. The total volume of liquid is kept as low as possible, and the concentration of the antimicrobials is kept high. In some embodiments, the volume per reservoir containing an antimicrobial is 1 ml. In some embodiments, the volume per reservoir containing an antimicrobial is 0.5 ml. In some embodiments, the volume per reservoir containing an antimicrobial compound is 0.1 ml.

In some embodiments the antimicrobials are solvated in the master cartridge in an aqueous solvent. In some embodiments the antimicrobials are solvated in the master cartridge in an organic solvent. Examples or organic solvents include but are not limited to dimethyl sulfoxide (DMSO), dimethyl formamide (DMF), ethanol, methanol, acetone, and N-methyl-2-pyrrolidone. In some embodiments a buffered aqueous solvent is used, for example, phosphate buffered saline (PBS).

In some embodiments, the antimicrobial is first solvated in a solvent or a solution having a pH greater than 8. In some embodiments, some antimicrobials are solvated using a solvent having a pH greater than 8.1, or 8.2, or 8.3, or 8.4 or 8.5 or 8.6 or 8.7 or 8.8, or 8.9, or greater than pH 9.0. In some embodiments an antimicrobial is first solvated in a solvent or a solution having a pH less than 7. In some embodiments some antimicrobials are solvated using a solvent having a pH less than 7, or less than 6 or less than 5 or less than 4 or less than 3. In some embodiments the antimicrobial is first solvated in an organic solvent. In some embodiments, the antimicrobial is first solvated using a first volume of a suitable solvent, and the remaining volume is made up with an aqueous solvent, or with water in order to achieve the desired concentration.

This approach requires a liquid handler to aliquot the antibiotics from the master cartridge or plate to “patient” or “daughter” cartridge or plates. The antibiotics may thus be present in the master plate at concentrations that are a multiple of the concentrations required in patient cartridges or plates. For example, for a daughter dilution series of 16 μg/mL, 8 μg/mL, 4 μg/mL, 2 μg/mL, 1 μg/mL, and 0.5 μg/mL the master plate may comprise concentrations of 320 μg/mL, 160 μg/mL, 80 μg/mL, 40 μg/mL, 20 μg/mL, and 10 μg/mL, such that each well is diluted 20-fold in concentration in transfer from master-to-daughter patient cartridges.

The master plate may also be designed to require fewer dilutions, conserving wells. This may be advantageous for utilizing 96-well master plates for use with 384- or 1536-well daughter plates, which may have advantages for high-volume plate filling. For example, for a daughter dilution series of 16 μg/mL, 8 μg/mL, 4 μg/mL, 2 μg/mL, 1 μg/mL, and 0.5 μg/mL the master plate may only comprise concentrations of 320 μg/mL, 40 μg/mL, and 10 μg/mL. In this case, the daughter plates would be filled with two different dilutions for each master concentration, 20-fold and 40-fold.

Additionally, greater numbers of dilutions in transfers may be performed. In the extreme case, each antibiotic may only comprise a single concentration, which is aliquoted into the appropriate daughter plate dilution range by the liquid handler.

In some embodiments, a master cartridge comprises three 96 well plates, one comprising antimicrobials for gram negative bacteria only, one comprising antimicrobials for gram positive bacteria only and one comprising the broad spectrum antimicrobials that work on both gram positive and gram negative bacteria (the broad spectrum plate). In some embodiments, master cartridge may comprise all antimicrobials laid out on the single master cartridge plate.

The transfer of antimicrobials from master-to-patient cartridge is accomplished by a liquid handler. Exemplary platforms include the Hamilton Nexus and Starlit and the Dynamic Devices Lynx. Other off-the-shelf or custom platforms comprising similar robotics and liquid handlers may also be utilized. These platforms may aliquot antibiotics, broth, and patient sample, therefore allowing daughter cartridges to “arrive” to the machine empty, greatly increasing storage and handling ease for laboratory customers. The liquid handlers may further enable antibiotic customization, such that only a subset of antibiotics is tested for specific patient samples. Alternatively, antibiotic selection/suppression may be made at the software level of the AST analyzer.

Additional benefits of the master-to-daughter antibiotic transfer approach the ability to accommodate antibiotics that are sparingly (or not at all) soluble in water. Solubilization for these agents may be enhanced through the use of detergents or other liquids or through the use of non-aqueous solvents. These may be present in the master cartridge itself and/or in reagent packs added to the liquid handler that prepares daughter plates.

In some embodiments, master cartridges can be designed such that antimicrobials derived from two or more different master cartridges are comprised on a patient cartridge.

In some embodiments the antimicrobials are lyophilized onto the master cartridge.

In some embodiments the antimicrobials are present in the master cartridge as dry powder.

In some embodiments the antimicrobials are present in a solution in high concentration.

Antimicrobials stored in master cartridge are at least greater than 20-fold concentrated than the minimal inhibitory concentration (MIC) for the antimicrobial for a target microbe. Antimicrobials are present in the master cartridge at a concentration that is at least greater than 30-fold, 40-fold, 50-fold, 60-fold, 70-fold, 80-fold, 100-fold or 200-fold or 500-fold concentration than the minimal inhibitory concentration (MIC) for the antimicrobial for a target microbe. In some embodiments the master cartridge comprises as high as 1000-fold the amount of each antimicrobial required to prepare one patient cartridge.

In some embodiments, each reservoir in a master cartridge contains greater than 1 microgram of the antimicrobial. In some embodiments, each reservoir in a master cartridge contains greater than 1 milligram of the antimicrobial. In some embodiments, each reservoir in a master cartridge contains greater than 10 milligrams of the antimicrobial. In some embodiments, each reservoir in a master cartridge contains greater than or equal to 100 milligrams of the antimicrobial. In some embodiments, each reservoir in a master cartridge contains greater than or equal to 1 gram of the antimicrobial. In some embodiments, each reservoir in a master cartridge contains greater than or equal to 10 grams of the antimicrobial. In some embodiments, each reservoir in a master cartridge contains as much as 100 grams of the antimicrobial. In some embodiments the antimicrobials in the master cartridge are stable through more than one freeze-thaw cycles. The high concentration of the antimicrobials in the master cartridge is such that one or more freeze thaw cycles cannot affect the integrity or functional efficacy of the antimicrobials.

In some embodiments, the master cartridge comprises 384 well microtiter plate.

In some embodiments the master cartridge comprises one or more seals. In some embodiments, an outer seal isolates the cartridge from the surrounding. This may be particularly beneficial for transportation and maintaining sterility. In some embodiments the master cartridge comprises an inner seal covering the one or more reservoirs.

In some embodiments the master cartridge is used to set up a multiplex AST assay for performing a plurality of different assays sharing an incubation period, wherein each assay comprises a microorganism growth assay in the presence of one or more antimicrobials, wherein the plurality of different assays are performed on a patient cartridge comprising one or more reservoirs and one or more antimicrobial compounds, wherein the antimicrobials in the cartridge are transferred to the patient cartridge from a master cartridge that contains each antimicrobial compound present at sufficient mass such that solvation in 0.1 mL of suitable solvent yields an antimicrobial concentration >10-fold higher than the highest desired testing concentration; and determining antimicrobial susceptibility of the one or more microorganisms based on relative microorganism growth.

The master cartridge is not brought in contact with any patient sample, and therefore can be reused to set up multiple rounds of such assays at different times.

### Patient Cartridge

In certain cases it may be preferable to have a patient cartridge with antimicrobials dried or frozen solvated at amounts appropriate for direct testing with samples comprising microorganisms derived from patient samples. Existing methods for performing automated AST interrogate reservoirs multiple times throughout the incubation period of the sample under test with antimicrobials comprised in the patient cartridge. This approach produces a growth curve that can be utilized to determine an MIC or growth/no-growth parameter for antimicrobials under test. However, the need for repetitive testing, combined with the throughput requirements of typical hospital clinical microbiology laboratories (for example, up to 170 ASTs per day for a hospital with 1034 beds), limit the number of reservoirs per cartridge.

New approaches for automated AST, such as those described in earlier filings U.S. Pat. No. 9,834,808; pending U.S. application Ser. No. 15/717,569 filed on Sep. 27, 2017, pending U.S. Provisional Application 62/524,972, filed on Jun. 26, 2017; published PCT Application WO2017185012, filed on Apr. 21, 2017 and pending PCT Application PCT/US17/68306 filed on Dec. 22, 2017; all of which are incorporated by reference herein, may perform AST with fewer reservoir interrogations. In particular, such methods may not require growth curves to report MICs. This advancement may enable patient cartridges with ≥150 reservoirs to be utilized with automated AST platforms, such as those described in our above mentioned Applications and Patent.

The number of reservoirs may be determined by considering the number of antimicrobials to be tested multiplied by the number of desired dilutions. In some embodiments, the required number of dilutions of one antimicrobial is different from that of another. As shown in Table 2 depicting calculations for an exemplary patient cartridge, the required number of inoculation reservoirs can be derived by calculating the sum of the number of dilutions necessary for all antimicrobials, which comprises (a) antimicrobials known to be effective against both gram positive and gram negative bacteria (“Broad Spectrum”, Combo) (x), (b) antimicrobials known to be effective against gram positive (y), and (c) antimicrobials known to be effective against gram negative bacteria (z) (=x+y+z). There are 51 different antimicrobials selected to be tested here. At least 128 reservoirs are required for the Broad Spectrum antimicrobials, at least 115 reservoirs for antimicrobials against gram negative and at least 102 reservoirs for antimicrobials against gram positive antimicrobials The exemplary patient cartridge in Table 2 therefore comprises at least 243 reservoirs for gram-negative bacteria and at least 230 reservoirs for gram-positive bacteria. In alternative embodiments all dilutions may be prepared on a single plate for all bacteria, comprising 345 reservoirs.

TABLE 2
Patient cartridge reservoirs for inoculation

| | | | Required | Required | Number |
|---|---|---|---|---|---|
| Type | Antibiotic | Abbreviation | Min | Max | Dilutions |
| Broad | Amikacin | AMK | 0.5 | 128 | 9 |
| Spectrum | | | | | |
| Broad | Ampicillin | AMP | 0.0625 | 64 | 11 |
| Spectrum | | | | | |
| Broad | Ciprofloxacin | CIP | 0.03125 | 8 | 9 |
| Spectrum | | | | | |
| Broad | Ceftriaxone | CRO | 0.25 | 16 | 9 |
| Spectrum | | | | | |
| Broad | Ceftazidime/Avibactam | CZA | 2/4 | 32/4 | 5 |
| Spectrum | | | | | |
| Broad | Doxycycline | DOX | 1 | 32 | 6 |
| Spectrum | | | | | |
| Broad | Cefoxitin | FOX | 1 | 32 | 6 |
| Spectrum | | | | | |
| Broad | Gentamicin | GEN | 0.25 | 32 | 8 |
| Spectrum | | | | | |
| Broad | Levofloxacin | LVX | 0.25 | 16 | 7 |
| Spectrum | | | | | |
| Broad | Minocycline | MNC | 0.5 | 32 | 9 |
| Spectrum | | | | | |
| Broad | Moxifloxacin | MXF | 0.5 | 16 | 6 |
| Spectrum | | | | | |
| Broad | Nitrofurantoin | NIT | 4 | 256 | 7 |
| Spectrum | | | | | |
| Broad | Ampicillin/sulbactam | SAM | 1/0.5 | 64/32 | 7 |
| Spectrum | | | | | |
| Broad | Trimethoprim/Sulfamethoxazole | SXT | 0.5 | 64 | 8 |
| Spectrum | (1:20) | | | | |
| Broad | Tetracycline | TET | 0.25 | 32 | 8 |
| Spectrum | | | | | |
| Broad | Tigecycline | TGC | 0.015625 | 16 | 11 |
| Spectrum | | | | | |
| Broad | Tobramycin | TOB | 0.125 | 32 | 9 |
| Spectrum | | | | | |
| GramNEG | Amoxicillin/Clavulanic Acid | AMC | 1/0.5 | 64/32 | 7 |
| GramNEG | Aztreonam | ATM | 1 | 64 | 7 |
| GramNEG | Ceftolozane-Tazobactam | C/T | 0.25/4 | 64/4 | 9 |
| GramNEG | Ceftazidime | CAZ | 0.5 | 32 | 7 |
| GramNEG | Ceftazidime/Clavulanate | CAZ/CLV | 0.5/4 | 0.5/4 | 1 |
| GramNEG | Cefuroxime | CFX | 1 | 64 | 7 |
| GramNEG | Cefazolin | CFZ | 0.25 | 32 | 8 |
| GramNEG | Cefpodoxime | CPD | 0.5 | 16 | 6 |
| GramNEG | Colistin | CST | 0.125 | 8 | 7 |
| GramNEG | Cefotaxime | CTX | 0.25 | 64 | 9 |
| GramNEG | Cefotaxime/Clavulanate | CTX/CLV | 0.5/4 | 0.5/4 | 1 |
| GramNEG | Doripenem | DOR | 0.0625 | 8 | 10 |
| GramNEG | ErtaPenem | ERT | 0.03125 | 16 | 10 |
| GramNEG | Cefepime | FEP | 0.25 | 32 | 10 |
| GramNEG | Cefepime/Clavulanate | FEP/CLV | 1/10 | 1/10 | 1 |
| GramNEG | Imipenem | IMP | 0.125 | 32 | 9 |
| GramNEG | Meropenem | MEM | 0.125 | 16 | 8 |
| GramNEG | Piperacillin/Tazobactam | TZP | 4/4 | 256/4 | 7 |
| GramPOS | Azithromycin | AZM | 0.25 | 16 | 7 |
| GramPOS | Clindamycin | CLI | 0.03125 | 16 | 10 |
| GramPOS | Clindamycin/Erythromycin | CLI/ERY | 0.5/1 | 0.5/4 | 1 |
| GramPOS | Ceftaroline | CPT | 0.03125 | 8 | 9 |
| GramPOS | Daptomycin | DAP | 0.0625 | 8 | 10 |
| GramPOS | Erythromycin | ERY | 0.125 | 16 | 8 |
| GramPOS | Gentamicin HL | GENHL | 500 | 500 | 1 |
| GramPOS | Linezolid | LNZ | 0.25 | 16 | 7 |
| GramPOS | Mupirocin (HL) | MUPHL | 256 | 256 | 1 |
| GramPOS | Oxacillin | OXA | 0.03125 | 8 | 9 |
| GramPOS | Benzylpenicillin (Penicillin G) | PEN | 0.03125 | 16 | 10 |
| GramPOS | Quinupristin/Dalfopristin | QNP/DFP | 0.125 | 8 | 7 |
| | (30:70) | | | | |
| GramPOS | Rifampin | RIF | 0.25 | 8 | 6 |
| GramPOS | Streptomycin HL | STPHL | 1000 | 1000 | 1 |
| GramPOS | Tedizolid | TDZ | 0.125 | 4 | 6 |
| GramPOS | Vancomycin | VAN | 0.25 | 64 | 9 |

A 384 well cartridge format is described herein, and was shown to yield reproducible and reliable MIC data. Usually a plate having greater than 96 wells is not preferred because of smaller well capacity, and especially evaporation of the solution could affect data outcome when working with a small volume of liquid. Additionally it was observed that there occurs an uneven loss of solution based on the position of a well on the plate. Wells at the periphery undergo greater level of evaporation than the wells toward the center of the well, as shown in the simple test depicted in an exemplary test herein. It was therefore surprising and unexpected, that the AST assay would be successful when performed in a 384 well plate. On the contrary, data from 384 well plate assays were highly reliable.

### Antimicrobials

Any antimicrobial can be adapted to the system provided in the disclosure. Examples include but are not limited to Amikacin, Amikacin-fosfomycin, Amoxicillin, Amoxicillin-clavulanate, Ampicillin, Ampicillin-sulbactam, Azithromycin, Azlocillin, Aztreonam, Aztreonam-avibactam, Besifloxacin, Biapenem, Cadazolid, Carbenicillin, Cefaclor, Cefamandole, Cefazolin, Cefdinir, Cefditoren, Cefepime, Cefepime-tazobactam, Cefetamet, Cefixime, Cefmetazole, Cefonicid, Cefoperazone, Cefotaxime, Cefotetan, Cefoxitin, Ceftolozane-tazobactam, Cefpodoxime, Cefprozil, Ceftaroline, Ceftaroline-avibactam, Ceftazidime, Ceftazidime-avibactam, Ceftazidime-avibactam, Ceftibuten, Ceftizoxime, Ceftobiprole, Ceftolozane-tazobactam, Ceftriaxone, Cefuroxime, Cephalothin, Chloramphenicol, Cinoxacin, Ciprofloxacin, Clarithromycin, Clinafloxacin, Clindamycin, Colistin, Dalbavancin, Daptomycin, Delafloxacin, Dirithromycin, Doripenem, Doxycycline, Enoxacin, Eravacycline, Ertapenem, Erythromycin, Faropenem, Fidaxomicin, Finafloxacin, Fleroxacin, Fosfomycin, Fusidic acid, Garenoxacin, Gatifloxacin, Gemifloxacin, Gentamicin, Gepotidacin, Grepafloxacin, Iclaprim, Imipenem, Imipenem-relebactam, Kanamycin, Lefamulin, Levofloxacin, Levonadifloxacin, Linezolid, Linopristin-flopristin, Lomefloxacin, Loracarbef, Mecillinam, Meropenem, Methicillin, Mezlocillin, Minocycline, Moxalactam, Moxifloxacin, Nafcillin, Nalidixic acid, Netilmicin, Nitrofurantoin, Norfloxacin, Ofloxacin, Omadacycline, Oritavancin, Oxacillin, Penicillin, Piperacillin, Piperacillin-tazobactam, Plazomicin, Polymyxin B, Quinupristin-dalfopristin, Razupenem, Rifampin, Solithromycin, Sparfloxacin, Sulfisoxazole, Sulopenem, Tedizolid, Teicoplanin, Televancin, Telithromycin, Tetracycline, Ticarcillin, Ticarcillin-clavulanate, Tigecycline, Tobramycin, Trimethoprim, Trimethoprim-sulfamethoxazole, Trospectomycin, Vancomycin, Aculeacin A, Amphotericin B, Caspofungin, Clotrimazole, Fluconazole, Flucytosine, 5-Fluorocytosine, Griseofulvin, Itraconazole, Ketoconazole, Nystatin, Sordarin, Terbinafine, Voriconazole and theirs salts or hydrates.

In some embodiments the antimicrobials are chemically synthesized molecules. In some embodiments the antimicrobials are chemical compounds. In some embodiments the antimicrobials are biomolecules such as peptides. In some embodiments the antimicrobials are biomolecules such as nucleotides or amino acids. In some embodiments the antimicrobials are biologically molecules. In some embodiments the antimicrobials are antibodies.

The antimicrobials can be stable at room temperature. In some embodiments the antimicrobials are not stable at room temperature in solubilized form. In some embodiments the antimicrobials are susceptible to degradation when stored at a higher temperature, such as room temperature. Several activity assays are available to measure the half-life of an antimicrobial under any conditions over any period of storage. Such methods of assay are well known to one of skill in the art and are not covered in the present disclosure. Creating and storing master cartridges at high antimicrobial concentrations or as dry powder extends the half-life of an antimicrobial. In some embodiments, antimicrobials are stable through multiple freeze-thaw cycles when stored in a master cartridge. In some embodiments the antimicrobials present in dry form, and are solvated in a suitable solvent and/or further diluted. Solvation fluid can be an organic solvent, or an inorganic solvent, acidic or basic in nature. Further dilution is carried out in water. Table 3 provides the suitable solvents for common antimicrobials necessary for AST assays.

TABLE 3
Antimicrobial solvents.

| Drug | Solvent |
|---|---|
| Amikacin | Water |
| Amoxicillin | 3 mL DMSO, 0.01M Phosphate Buffer pH |
| | 8.0 at time of fill |
| Ampicillin | 0.1M Phosphate Buffer pH 8.0 |
| Avibactam | Water |
| Avibactam | Water |
| Azithromycin | 95% Ethanol |
| Aztreonam | Water |
| Cefazolin | 0.1M Phosphate Buffer pH 6.0 |
| Cefepime | 5 mL DMSO, 0.01M Phosphate Buffer pH |
| | 8.0 at a time to fill |
| Cefotaxime | Water |
| Cefoxitin | Water |
| Cefpodoxime | 0.1% Sodium bicarbonate |
| Ceftaroline | 30% DMSO/70% saline |
| Ceftazidime | Water |
| Ceftazidime | Water |
| Ceftolozane | Water |
| Ceftriaxone | Water |
| Cefuroxime | 0.1M Phosphate Buffer pH 6.0 |
| Ciprofloxacin | 5 mL H2O, add 1 mL 5N NaOH, and 4 mL |
| | H2O |
| Clavulanic Acid | 0.1M Phosphate Buffer pH 6.0 |
| Clindamycin | Water |
| Colistin | Water |
| Daptomycin | Water |
| Doxycycline | Water |
| Ertapenem | 0.01M Phosphate Buffer pH 7.2 |
| Erythromycin | 95% Ethanol |
| Gentamicin | Water |
| Imipenem | 0.01M Phosphate Buffer pH 7.2 |
| Levofloxacin | 3 mL H2O, add 1 mL 5N NaOH, and 1 mL |
| | H2O |
| Linezolid | 95% Ethanol |
| Meropenem | Water |
| Minocycline | Water |
| Moxifloxacin | Water |
| Nitrofurantoin | DMSO |
| Norfloxacin | 3 mL H2O, add 2 mL 5N NaOH, and 2 mL |
| | H2O |
| Oxacillin | Water |
| Penicillin | Water |
| Piperacillin | Water |
| Quinupristin/Dalfopristin | Water |
| Rifampin | Methanol |
| Sulbactam | Water |
| Sulfamethoxazole | Acetone |
| Tazobactam | Water |
| Tedizolid | DMSO |
| Teicoplanin | Water |
| Tetracycline | 3 mL MeOH + 2 mL water. 5N NaOH after |
| | diluting |
| Tigecycline | Water |
| Tobramycin | Water |
| Trimethoprim | Water |
| Vancomycin | Water |

An assay setup comprises preparation of patient (target) cartridge by dispensing antimicrobials were from the master cartridge or intermediate serial dilution cartridges into one or more 384 reservoir patient cartridge, each antimicrobial in about 7 serial dilutions in triplicate, and covering the dynamic range of each antimicrobial that is known to be effective and therefore should be reported. The dilution range included the expected minimum inhibitory concentration (MIC) for each antimicrobial. But most importantly, dilution ranges, that is, antimicrobial concentrations beyond the range known to be effective are included in the patient cartridge as per the present invention.

The remaining reservoirs of the 384 well patient cartridge are utilized for setting up test controls: a no-antimicrobial control (negative control) was included for each antimicrobial compound; and a positive control was included for each antimicrobial set, where a microorganism that is not susceptible to the antimicrobial was added to the well. Each control set was also dispensed at least in duplicate per 384 well cartridge. Additional test controls may be included as deemed necessary by one of skill in the art. Equal amount of a patient sample was dispensed to each of the wells in the cartridge, except in the wells designated for no-sample control, if included. The patient cartridge was ready for determination of susceptibility of microbes from the patient sample to the twelve antimicrobials at the range of concentrations applied, simultaneously. Multiple such plates can be set up in parallel for testing samples from multiple patients, each patient sample per plate. An AST assay was performed on the prepared patient cartridges.

In general, AST assays are performed using 24 well-96 well plates. As disclosed herein, in some embodiments the AST assay is performed in 384 well plates. Applicants show that high quality AST results could be obtained using a 384 well plate assay. Since the volume of each reservoir in a 384 well plate is considerably smaller than the 96 well plate, reagents are proportionately scaled down for the assay, thereby posing considerable uncertainty of the assay and data reliability. For example, evaporation could affect the concentrations of the solutions within, and the rate of bacterial growth or a chemical reaction. Surprisingly, it was found that the assay method used as per the invention led to successful AST assays and reliable results when performed on a 384 well plate.

### Diagnostic or Therapeutic Applications

The cartridges and methods described herein can be effective in diagnosing the nature of a microbial population in the biological sample from a subject. The subject can be a human patient. The subject can also be a non-human animal. The biological sample is obtained from the patient for analysis. The biological sample can be selected from a group consisting of blood, plasma, blood component, sputum, urine, an exudate, nasal swab, vaginal swab, throat swab, sweat, eye discharge or tissue homogenate. Information regarding susceptibility to one or more antimicrobial in qualitative and quantitative assessment is obtained as a result of the product and methods described herein.

The present invention may be used to treat various diseases, disorders and conditions. Determination of an antimicrobial which is effective against one or more microbe in a patient during a short period of investigation as well as obtaining an MIC value positively impact treatment decisions by a practitioner. The present invention facilitates such outcome in a number of ways. For example, availability of master cartridge could overcome shipping distance barriers, weight restrictions, temperature and stability concerns, and therefore makes an antimicrobial screening endeavor possible at a location of a microbial infection outbreak. Moreover, since multiple “daughter” cartridges can be generated from a master cartridge, the ability for large scale screening of both qualitative and quantitative nature using the patient sample directly, or with multiple patient samples simultaneously, obviates the necessity to identify the microorganism before starting an effective therapeutic approach without delay to the patient(s). The approach aids determination of effective therapeutic dose of the antimicrobial of choice. Thirdly, the batch to batch variability is reduced using the master cartridge approach, allowing reproducibility of diagnostic and therapeutic decisions.

In some embodiments, master cartridges are prepared for downstream use in analyzing antimicrobials for certain indications, where a practitioner of the art would expect a certain group of antimicrobials to work. A close comparison of such antimicrobials for selection of the most effective antimicrobial for a given indication would require such antimicrobials to be selective present in a single set. Therefore, by carefully selecting antimicrobials that can be included in a master cartridge, several platform antimicrobial AST arrays can be custom-generated as per necessity and demand in the field.

### EXAMPLES

While certain articles, compositions and methods have been described with specificity in accordance with certain embodiments, the following examples serve only to illustrate embodiments of the invention and are not intended to limit the same.

### Example 1: Freezer Space Usage in Multiplex Assays Using Master Cartridge

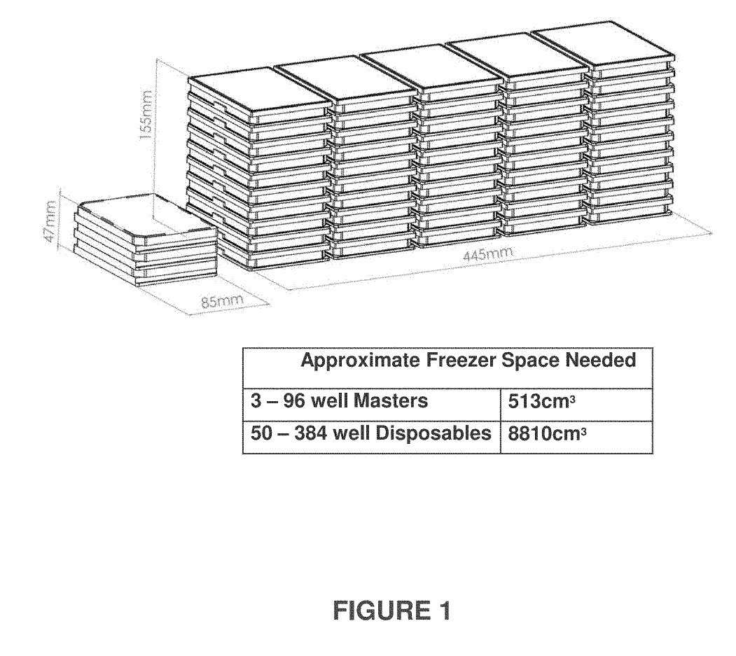

This example depicts an estimate of freezer space saved by shipping and storing the AST assay cartridges in a master cartridge format. A master cartridge as per the invention is shipped and stored in freezer as an alternative of the commonly prevalent procedure of shipping and storing test cartridges (i.e., patient cartridges) until use. A master cartridge comprising antimicrobials in high concentration and sufficient mass to In this example, a master cartridge is a stack of three 96 well plates, which require a space of 513 cm³. The master plate stack requires a footprint of 128mm×85mm×47mm. A master plate can generate a daughter set of patient cartridge of fifty plates, each plate having 384 reservoirs (wells). Therefore the master cartridge is equivalent to fifty 384 disposable well plates, which have a stacked calculated footprint of 8810cm³ (FIG. 1). The daughter plates are generated from the master cartridge on the day of the assay and therefore are dispensed once the assay is complete. Therefore, using a master cartridge reduces a shipping and freezer storage space by 17 fold.

### Example 2. Layout of Antimicrobials on a Master Cartridge

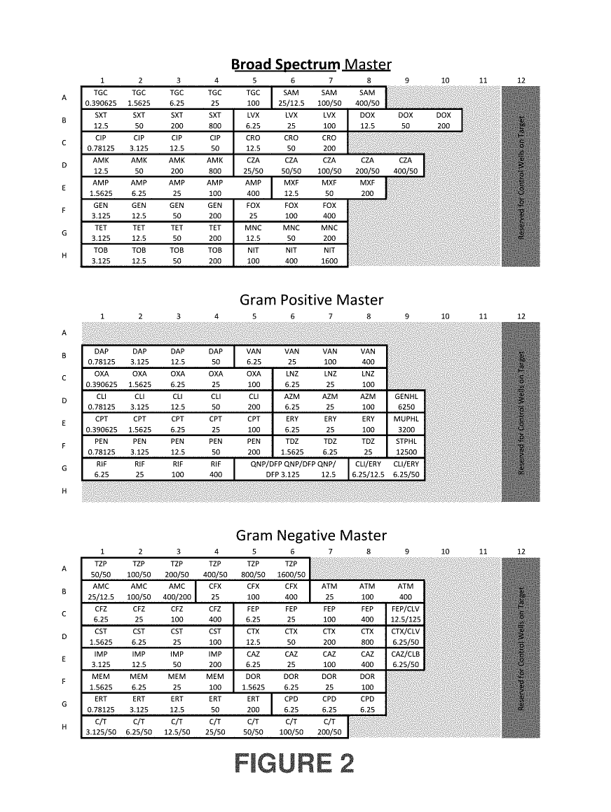

This example depicts a layout of antimicrobials on a master cartridge. In this example, three 96 well plates was used for master cartridge as shown in FIG. 2. Each master cartridge contains three types of antimicrobials: (1) ones that are “broad spectrum” antimicrobials, effective against both (the term “combo” is often used interchangeably herein with broad spectrum to designate this category) (2) ones that are known to be effective against gram negative bacteria, and (3) ones that are known to be effective against gram positive bacteria. Accordingly as shown in FIG. 2, plate comprising the Broad Spectrum antimicrobials, a plate comprising the gram negative antimicrobials and a plate comprising the gram positive antimicrobials are laid out. The master cartridge comprises high concentration of each antimicrobial (at least greater than five-fold of the highest concentration to be tested). For example, highest concentration of ampicillin recommended for testing in is 64 μg/ml. The highest concentration of ampicillin present in the master plate is 400 μg/ml. Additionally, the master cartridge also comprises sufficient mass of each antimicrobial adapted to prepare multiple patient cartridge from a single master cartridge.

### Example 3. Preparation of Patient Cartridge

In this example a step by step set up of patient cartridge from master cartridge for performing an automated AST assay is provided.

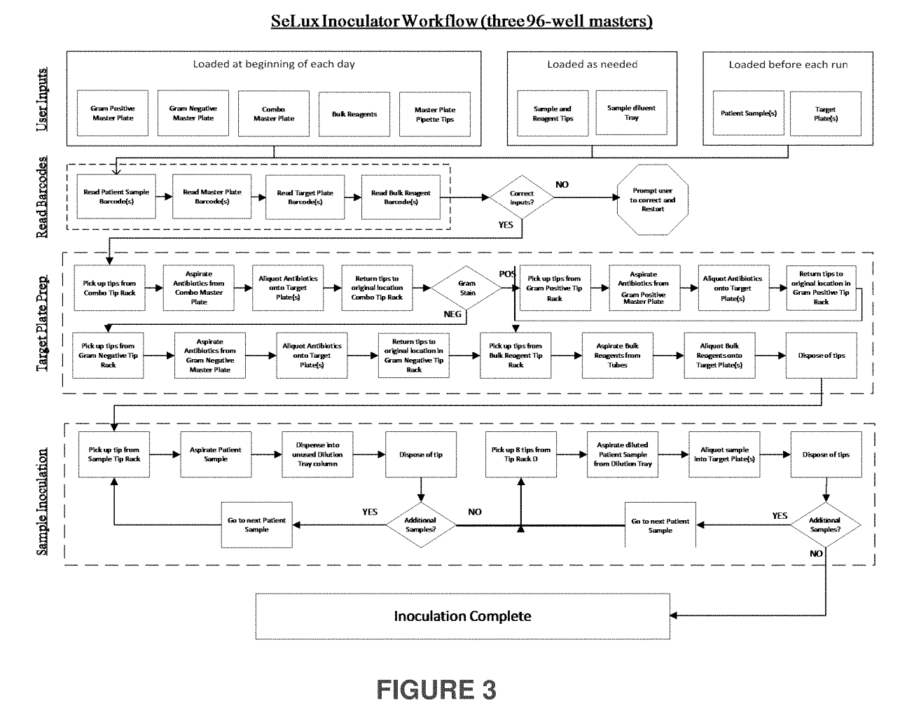

The process of using master cartridges to produce “patient cartridges” or target cartridges in clinical laboratory settings can be automated, using liquid dispensers available from multiple manufacturers, including Hamilton Company, Tecan, Hudson Robotics, etc. A schematic diagram of the inoculation workflow is given in FIG. 3.

First, three master plates are loaded by the user onto chilled plate holders (set to 4° C.). These consist of one Broad Spectrum, one Gram-Positive and one Gram-Negative antibiotic plate. Next the bulk reagents and pipette tips are loaded on tube rack. Master and bulk reagent pipette tips and sample dilution tray are reloaded every 12 samples. For every set of tests, up to four Patient Samples in tubes and up to four 384-well Target Plates (Patient plate) are loaded.

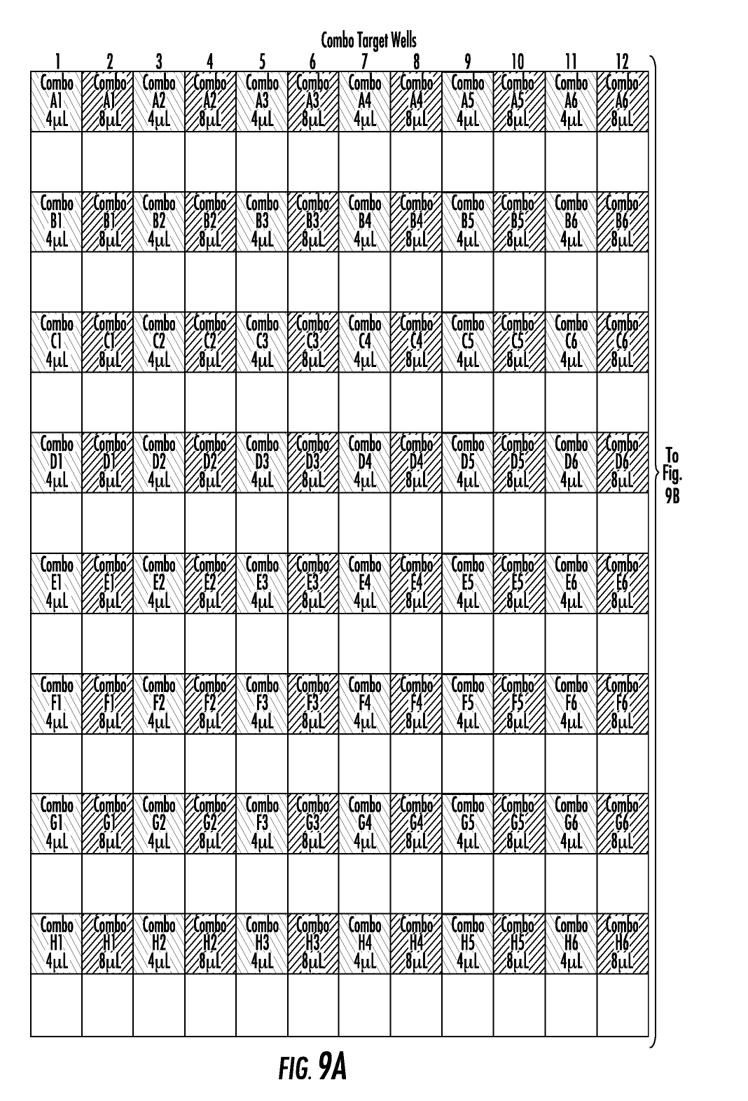

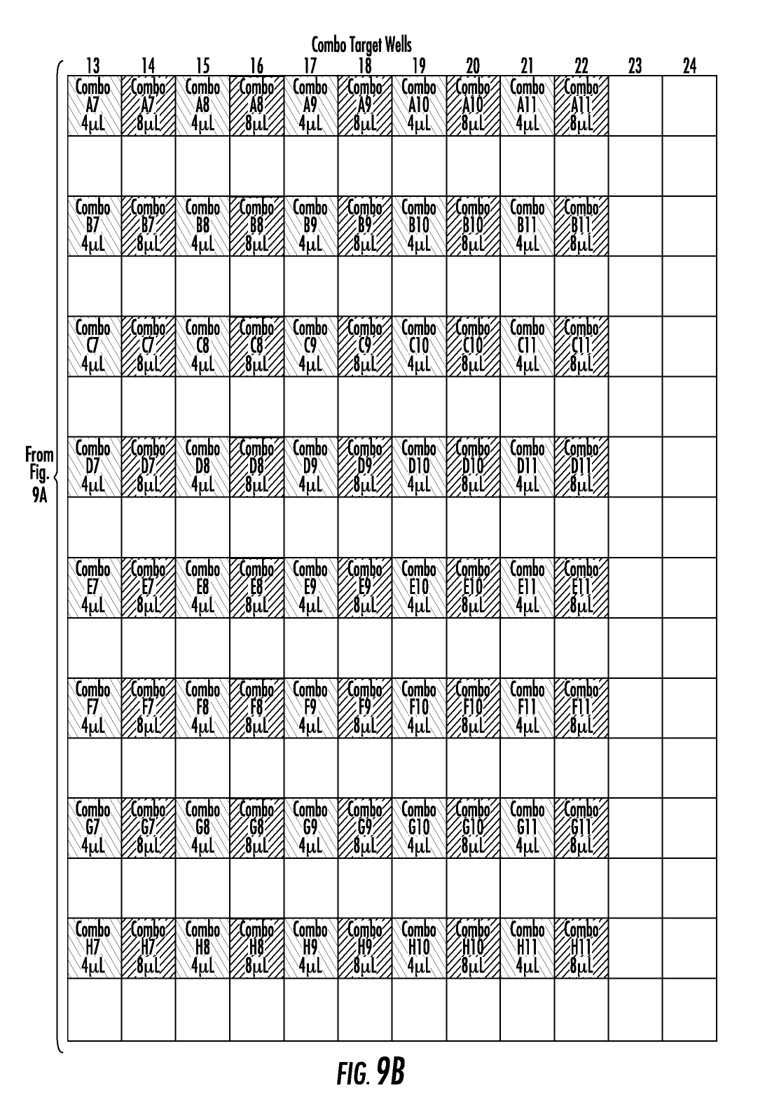

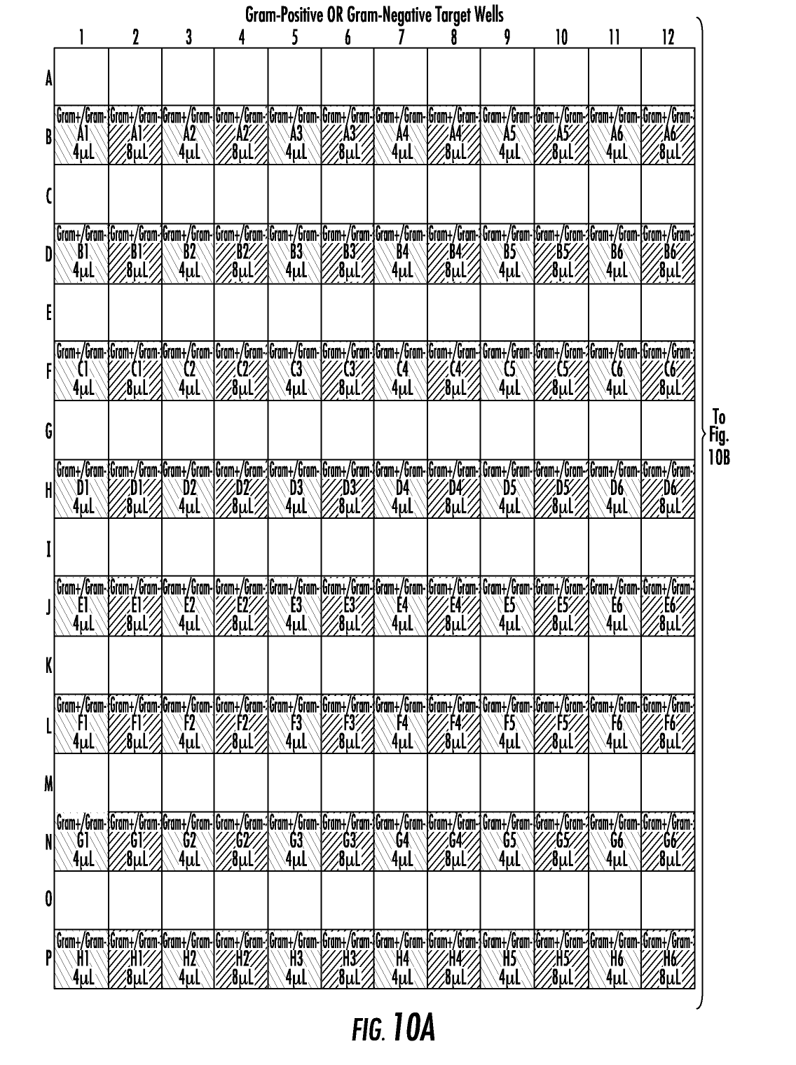

Patient (Target) Plate Preparation. Antibiotics are transferred from Broad Spectrum Master Plate to Target Plate(s). Antibiotics are “stamped” according to FIG. 9A-B. The inoculator tips should be placed back in the same position of the Broad Spectrum Tip Box for use on subsequent target plates, but should only be used for same antibiotics to avoid cross contamination.

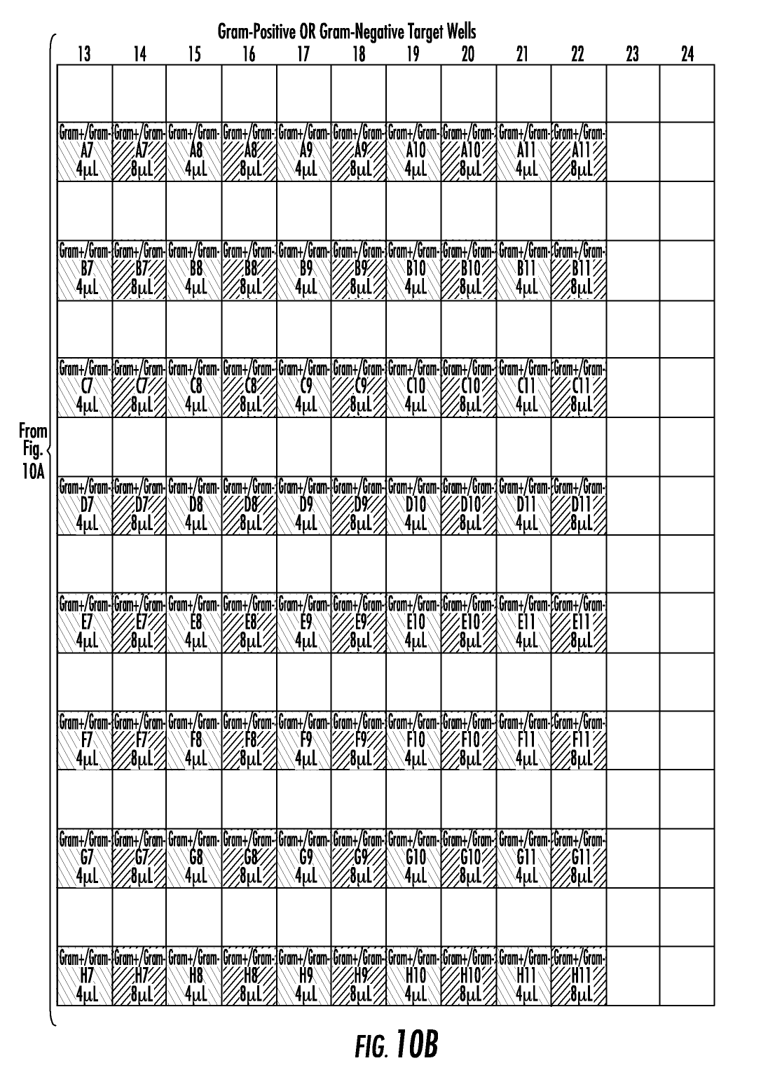

Antimicrobials are transferred from Gram-Positive OR Gram-Negative Master Plate to Target Plate(s) and the antibiotics are “stamped” according to FIG. 10A-B. The inoculator should place tips back in the same position of the correct tip box for use on subsequent target plates. Good care is taken such that any cross contamination is prevented.

Next, the bulk reagents are transferred to Target Plate(s) using Bulk Reagent tips, Target Plates according to Table 4.

Preparation of Patient Sample Dilutions. Using unused tips from the Sample Tip Box, 2004, of Patient Sample are transferred into an unused column of the Sample Dilution Tray. Used tips should be immediately disposed of to avoid cross contamination. The above steps a repeated for remaining patient samples.

Target Plate Inoculation with Patient Sample. Using new tips from the Sample Tip Box, 504, of diluted patient sample are transferred into Target Plate according to FIG. 9A-B, this is done with a “jet dispense” to avoid cross contamination. After the Patient Sample has been transferred to each well on the Target Plate, the tips should be disposed of to avoid cross contamination. The above steps are repeated for each additional Patient Sample and Target Plate.

The process can utilize one or more auxiliary cartridges in the machine. Care is taken that the auxiliary cartridges and the master cartridge are not inoculated with microorganisms. Serial dilutions of each antimicrobial compound in performed and dispensed on the sample patient cartridge.

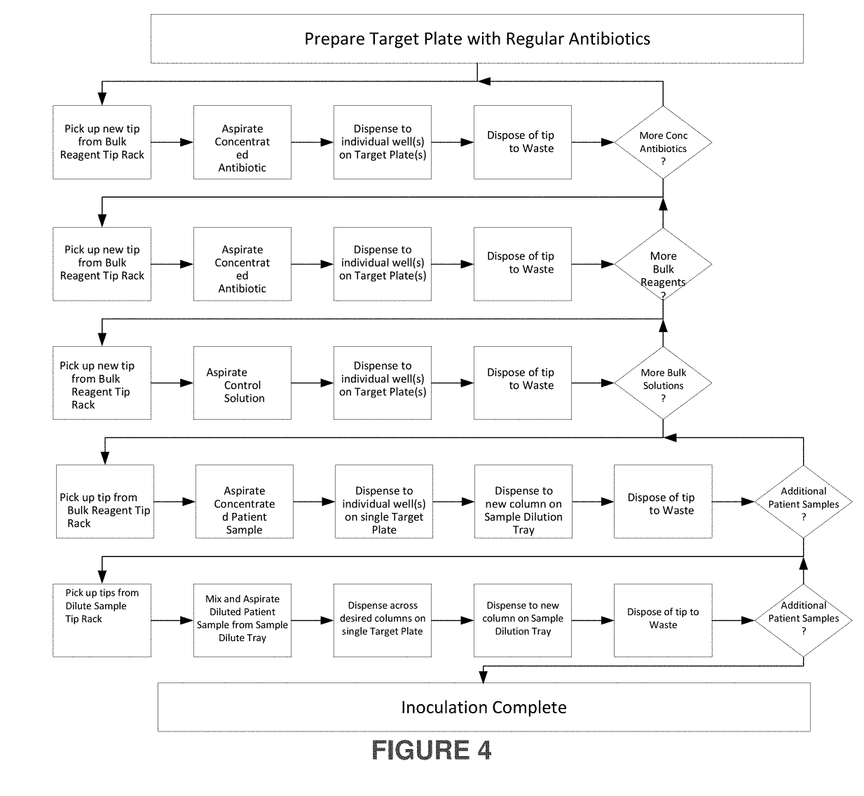

A schematic diagram of the individual well flow chart is given in FIG. 4.

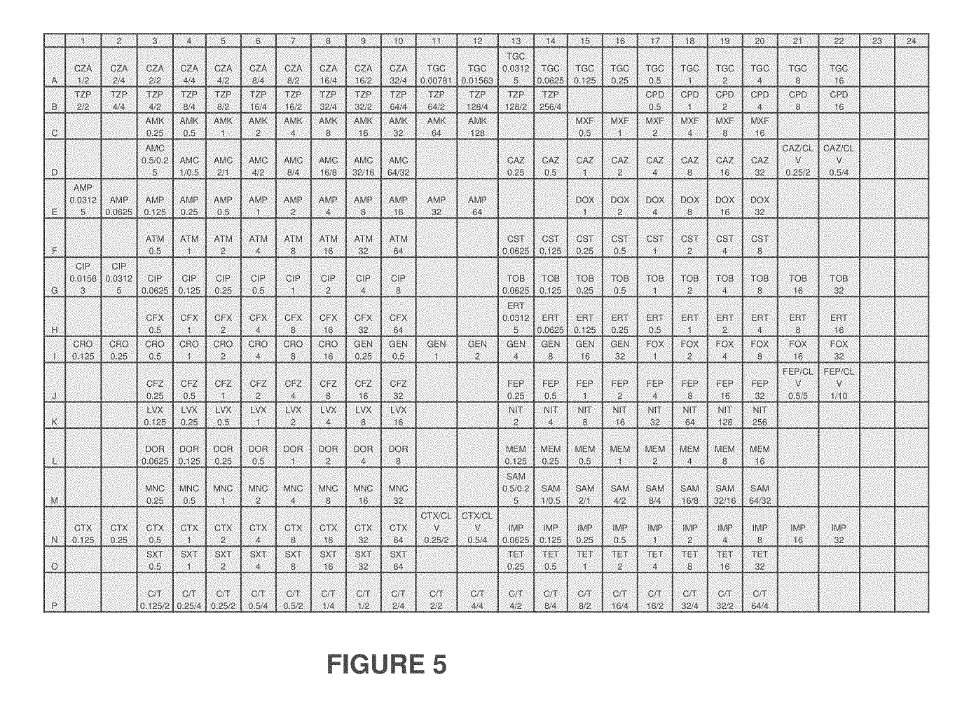

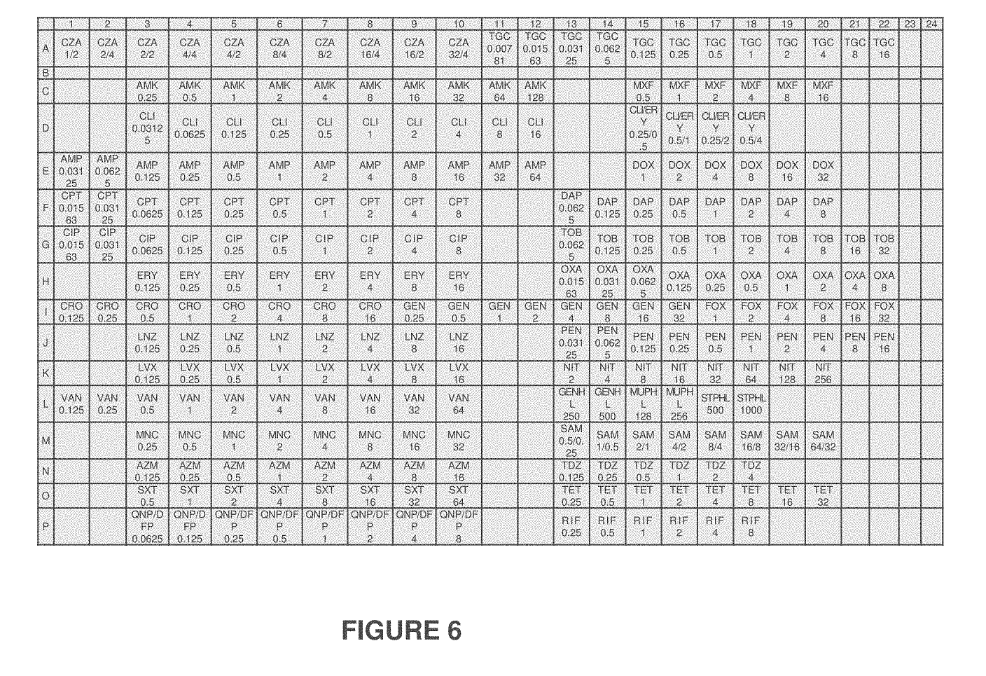

The final layout of a patient cartridge (also referred to as Target Plate) comprising antimicrobials against gram negative bacteria is depicted in FIG. 5. The final layout of a patient cartridge (also referred to as Target Plate) comprising antimicrobials against gram positive bacteria is depicted in FIG. 6.

For testing minimum inhibitory concentration (MIC) the range of serial dilutions of each antimicrobial dispensed is sufficient to cover the MIC over a dynamic range in several orders of magnitude. For qualitative susceptibility testing also, a sufficient dilution range is prepared on the sample patient cartridge, as per CLSI standards. These dilutions may be present in repetition and additional dilutions may be utilized. The concentrations of antimicrobial solutions in the sample patient cartridges are referred to as the “testing concentrations.” Testing concentrations represent all concentrations within the ranges for quality control or MIC interpretive criteria for a given antibiotic, as defined by the CLSI M100S Manual.

### Example 4. Rapid AST Performed in a 384-Well Plate Provides Similar Data to an Assay Performed in a 96-Well Plate

This example demonstrates successful AST assay on 384 well plate yielding high data reliability.

The antibiotics shown are vancomycin, daptomycin, ceftaroline and levofloxacin, of which the MICs obtained from the broth microdilution reference method for this strain were 0.5, 0.25, 0.12, and 0.25 μg/ml, respectively. A clinical isolate of S aureus was used. Data represents the TRF signal in RFUs. Graphs in the top row contain data from 384-well plates. Graphs in the bottom row contain data from 96-well plates.

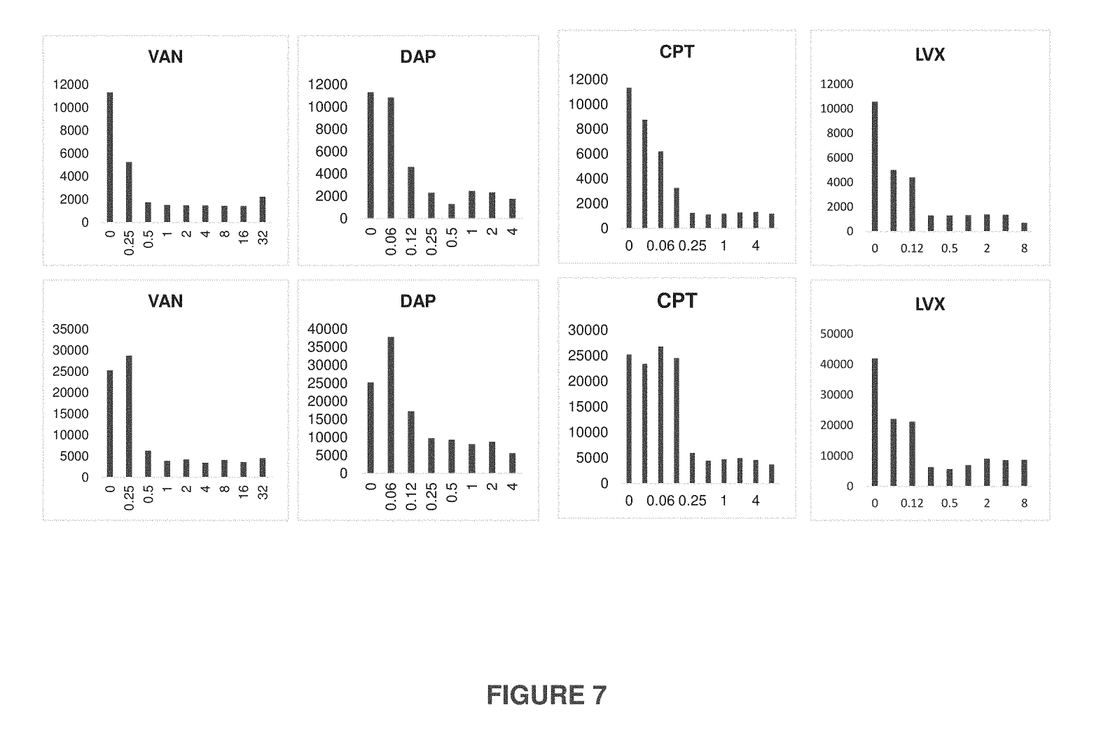

Shown in Table 5 and Table 6, MIC results for antimicrobial panels 1 and 2 respectively using a master cartridge format and a 384 well patient cartridge (column 3) agrees reliably with that run by standard 96 well AST assay plate (right hand column). FIG. 7 shows corresponding growth curves determining the MIC values of the antimicrobials on patient sample microbes. This shows that master cartridge with the 384 well plate format offers reliable results, in addition to the other advantages discussed herein.

TABLE 5
MIC results for antimicrobial panel 1

| | | MICs obtained | |
|---|---|---|---|
| | | from panel | MICs obtained |
| | Quality Control | made from | from 96-well |
| Antibiotic | Range | Master Plate | Antibiotic Panel |
| Ceftriaxone | 0.03-0.12 | ≤0.125 | ≤0.12 |
| Ceftazidime | 0.06-0.5 | <1 | 0.25 |
| Ampicillin- | 2-8 | 8 | 8 |
| Sulbactam | | | |
| Tobramycin | 0.25-1 | 0.5 | 1 |
| Amikacin | 0.5-4 | 4 | 4 |
| Ampicillin | 2-8 | 4 | 8 |
| Piperacillin- | 1-4 | ≤1 | 4 |
| tazobactam | | | |
| Levofloxacin | 0.008-0.06 | ≤0.25 | ≤0.06 |
| Cefepime | 0.015-0.12 | ≤0.25 | ≤0.03 |

TABLE 6
MIC results for antimicrobial panel 2

| | | MICs obtained | |
|---|---|---|---|
| | | from panel | MICs obtained |
| | Quality Control | made from | from 96-well |
| Antibiotic | Range | Master Plate | Antibiotic Panel |
| Linezolid | 1-4 | 2 | 2 |
| Ceftaroline | 0.12-0.5 | 0.5 | 0.25 |
| Tedizolid | 0.25-1 | 0.25 | 0.5 |
| Oxacillin | 0.12-0.5 | 0.5 | 0.5 |
| Rifampin | 0.004-0.015 | ≤0.25 | 0.004 |
| Ceftriaxone | 1-8 | 2 | 4 |
| Ceftazidime | 4-16 | 4 | 8 |
| Ampicillin | 0.5-2 | 1 | 1 |
| Levofloxacin | 0.06-0.5 | ≤0.25 | 0.25 |

### Example 5. Non-Uniform Volume Loss Detected in 384-Well Plates

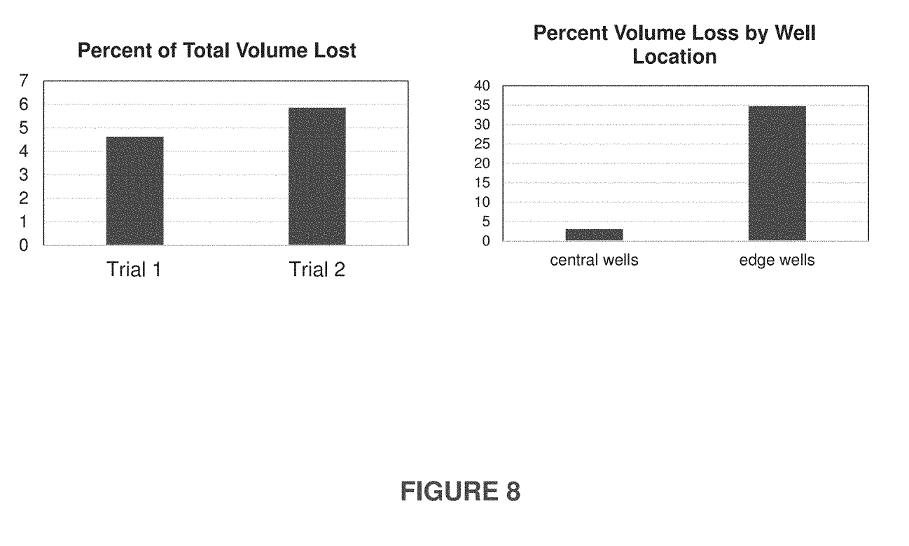

In order to test the rate of evaporation at various regions of the plate, 40 μl of water were added to each well in a 384-well plate. Plate masses were recorded and plates with lids were incubated shaking at 35° C., 150 rpm (Southwest Science Mini IncuShaker) overnight for approximately 18 hours. After incubation, plates were masses to determine total volume loss during incubation (FIG. 8, left panel). This data records the total loss of liquid volume on the entire plate. To determine the volume remaining in individual wells, micropipettes were used. The average percent loss of volume in at least 6 central or edge wells is reported (FIG. 8 right panel). This data indicates that the loss of liquid volume was non-uniform throughout the plate, with the wells in the periphery losing about 6 times more liquid volume than the ones in the center.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1-93. (canceled)

94. A patient cartridge for automated antimicrobial susceptibility testing suitable for inoculation with a microbial sample derived from a human sample, the cartridge comprising ≥150 reservoirs and ≥20 different antimicrobials, wherein:
(a) at least one assay quality control, where three or more reservoirs comprise no reagents;
(b) at least one assay quality control having three or more reservoirs comprising no reagents that influence microorganism growth; and
(c) the dilution ranges of at least 5 antimicrobials exceed the clinically relevant dilution ranges for the bacterial species by at least one antimicrobial concentration.

95. The patient cartridge of claim 94, comprising amikacin, ampicillin-sulbactam, aztreonam, cefepime, cefotaxime, ceftazidime, ceftazidime-avibactam, ceftriaxone, ciprofloxacin, gentamicin, imipenem, levofloxacin, meropenem, piperacillin-tazobactam, tetracycline, tobramycin, and trimethoprim-sulfamethoxazole.

96. The patient cartridge of claim 95, wherein the dilution ranges of a plurality of antimicrobials are clinically relevant for Enterobacteriaceae, Pseudomonas spp., and Acinetobacter spp.

97. The patient cartridge of claim 96, comprising ciprofloxacin, daptomycin, gentamicin, levofloxacin, linezolid, penicillin, tetracycline, and vancomycin.

98. The patient cartridge of claim 94, wherein the dilution ranges of a plurality of antimicrobials are clinically relevant for Staphylococcus spp. and Enterococcus spp.

99. The patient cartridge of claim 94, wherein the patient cartridge comprises a microtiter plate comprising 384 reservoirs.

100. The patient cartridge of claim 94, wherein the patient cartridge comprises a microtiter plate comprising 1536 reservoirs.

101. The patient cartridge of claim 94, wherein each reservoir comprises reservoir wall and a reservoir base, and wherein the reservoir walls for a plurality of reservoirs are opaque.

102. The patient cartridge of claim 94, wherein a plurality of the reservoirs allow >85% passage of light at 350 nm through the reservoir bases.

103. The patient cartridge of claim 101, wherein the reservoir walls and/or bases comprise polystyrene.

104. The patient cartridge of claim 103, wherein the polystyrene is untreated polystyrene.

105. The patient cartridge of claim 94, wherein the patient cartridge comprises antimicrobials in solid state.

106. A pouch comprising a patient cartridge of claim 105 and a desiccant, wherein the patient cartridge is sealed within the pouch comprising the desiccant.

107. The patient cartridge of claim 106, wherein the patient cartridge is stable for storage between 0° C. and 35° C.

108. The patient cartridge of claim 94, wherein the antimicrobials are frozen in solvated form.

109. A pouch comprising a patient cartridge of claim 94 and an adhesive cover, wherein the patient cartridge is sealed with the adhesive cover.

110. The patient cartridge of claim 94, further comprising a detachable lid.

111. The patient cartridge of claim 94, wherein an antimicrobial amount in a plurality of reservoirs is replicated in one or more additional reservoirs.

112. A method for automated antimicrobial susceptibility testing comprising:
(a) preparing a patient cartridge comprising about 384 reservoirs, wherein a first subset of the 384 reservoirs comprises one or more antimicrobials, by inoculating a second subset of the 384 reservoirs with a microorganism-comprising sample, and providing within the patient cartridge a plurality reservoirs for assay quality control, comprising a minimum of 3 reservoirs for negative control having no reagents; and a minimum or 3 reservoirs having no reagent that promote bacterial growth;
(b) incubating the cartridge under conditions promoting microorganism growth for a period between 2 and 24 hours;
(c) interrogating a plurality of reservoirs to assess microbial growth; and
(d) determining the MIC for the sample for a plurality of antimicrobials on the cartridge.

113. The method of claim 112, wherein a plurality of reservoirs on the cartridge are interrogated for growth between 1 and 5 times before the MIC is determined.

114. The method of claim 112, wherein a plurality of reservoirs on the cartridge are interrogated for growth between 1 and 4 times before the MIC is determined.

115. The method of claim 112, wherein a plurality of reservoirs on the cartridge are interrogated for growth between 1 and 3 times before the MIC is determined.

116. The method of claim 112, wherein no more than 98% of the reservoirs are utilized to provide MIC results.

117. The method of claim 112, wherein a minimum of 3 reservoirs are utilized to determine the incubation period when sufficient microbial growth has been achieved to initiate one or more assays for AST.

118. The method of claim 112, wherein one or more reagents are added to the patient cartridge after the incubation period.

119. The method of claim 112, wherein the concentration of patient sample inoculated in a reservoir within the second subset of reservoirs is different from the concentration of patient samples inoculated in a different reservoir within the same subset in the patient cartridge.

120. (canceled)

121. The method of claim 112, further comprising adding a chemical reagent solutions in a plurality of the reservoirs, wherein the chemical reagents solution comprise a molecule capable of undergoing a chemical reaction.
